(19) United States
(12) Patent Application Publication
(10) Pub. No.: US 2009/0111821 A1
(43) Pub. Date: Apr. 30, 2009

(54) AMINO 1,2,4-TRIAZOLE DERIVATIVES AS MODULATORS OF MGLUR5

(75) Inventors: Methvin ISAAC, Brampton (CA); Andreas WALLBERG, Molndal (SE)

Correspondence Address: BIRCH, STEWART, KOLASCH & BIRCH, LLP, P.O. BOX 747, FALLS CHURCH, VA, 22040-0747, US

(73) Assignee: AstraZeneca AB, Sodertalje (SE)

(21) Appl. No.: 12/258,114

(22) Filed: Oct. 24, 2008

Related U.S. Application Data
(60) Provisional application No. 60/982,956, filed on Oct. 26, 2007.

(51) Int. Cl. A61K 31/501; C07D 249/08; C07D 403/14; A61K 31/497; A61P 1/04; A61P 25/22; C07D 401/14; A61K 31/4196

(52) U.S. Cl. 514/252.05; 548/266.2; 544/238; 544/333; 544/405; 514/383; 514/255.05; 514/256

(57) ABSTRACT

The present invention is directed to novel compounds, to a process for their preparation, their use in therapy and pharmaceutical compositions comprising the novel compounds.

### FIELD OF THE INVENTION

The present invention is directed to novel compounds, their use in therapy and pharmaceutical compositions comprising said novel compounds.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of in mGluRs in intact mammalian neurons elicits one or more of the following responses: activation of phospholipase C; increase in phosphoinositide (PI) hydrolysis; intracellular calcium release; activation of phospholipase D; activation or inhibition of adenyl cyclase; increase or decrease in the formation of cyclic adenosine monophosphate (cAMP); activation of guanylyl cyclase; increase in the formation of cyclic guanosine monophosphate (cGMP); activation of phospholipase A₂; increase in arachidonic acid release; and increase or decrease in the activity of voltage- and ligand-gated ion channels. Schoepp et al., Trends Pharmacol, Sci. 14:13 (1993), Schoepp, Neurochem. Int. 24:439 (1994), Pin et al., Neuropharmacology 34:1 (1995), Bordi and Ugolini, Prog. Neurobiol 59:55 (1999).

Molecular cloning has identified eight distinct m-GluR subtypes, termed mGluR1 through mGluR8. Nakanishi, Neuron 13:1031 (1994), Pin et al., Neuropharmacology 34:1 (1995), Knopfel el al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders

Attempts at elucidating the physiological roles of Group I mGluRs suggest that activation of these receptors elicits neuronal excitation. Various studies have demonstrated that Group I mGluR agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, walking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastrointestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current—sub-units that are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Generally, compounds of the present invention have low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

Because of their physiological and pathophysiological significance, there is a need for new potent mGluR agonists and antagonists that display a high selectivity for mGluR subtypes, particularly the Group I receptor subtype, most particularly the mGluR5.

The object of the present invention is to provide compounds exhibiting an activity at metabotropic glutamate receptors (mGluRs), especially at the mGluR5 receptor. In particular, the compounds according to the present invention are predominantly peripherally acting, i.e. have a limited ability of passing the blood-brain barrier.

### DESCRIPTION OF THE INVENTION

The present invention relates to a compound of formula I:

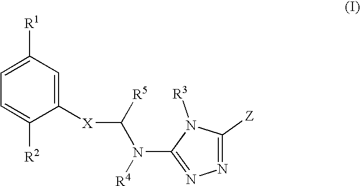

wherein

### X is

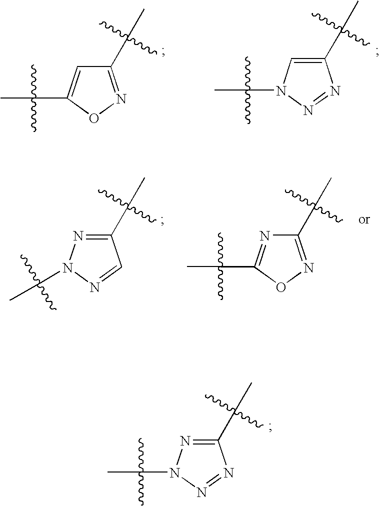

R¹ is methyl, halogen or cyano;
R² is hydrogen or fluoro;
R³ is C₁-C₃ alkyl or cyclopropyl;
R⁴ is C₁-C₃ alkyl or cyclopropyl;
R⁵ is hydrogen, C₁-C₃ alkyl or cyclopropyl;

### Z is

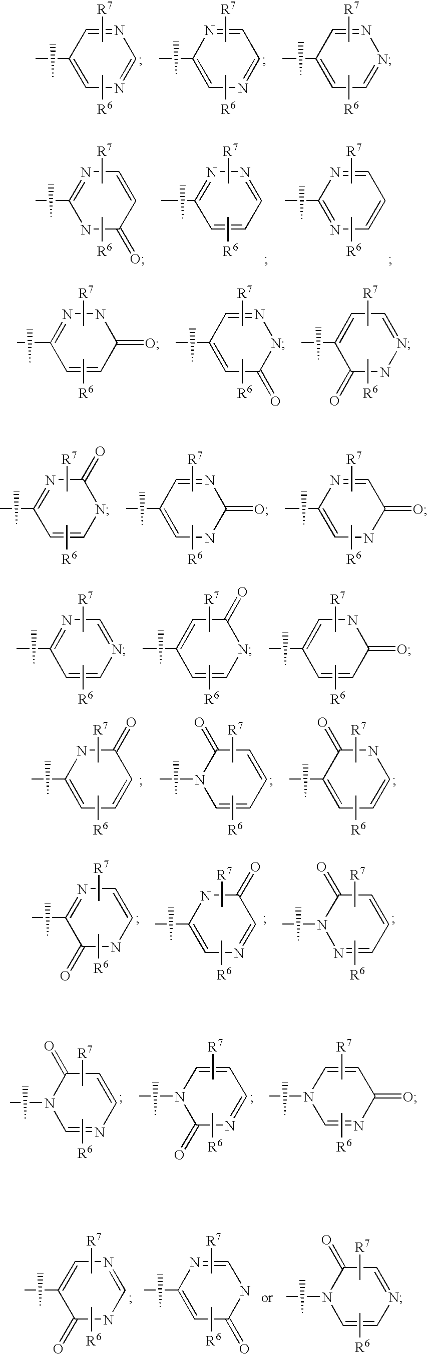

wherein
R⁶ is hydrogen, fluoro, C₁-C₃ alkyl or C₁-C₃ alkoxy;
R⁷ is hydrogen, fluoro, C₁-C₃ alkyl or C₁-C₃ alkoxy;
as well as pharmaceutically acceptable salts, hydrates, isoforms, tautomers and/or enantiomers thereof.

In one embodiment, R¹ is halogen.

In a further embodiment, R¹ is chloro.

In a further embodiment, R¹ is methyl.

In a further embodiment, R² is hydrogen.

In a further embodiment, R³ is methyl or cyclopropyl.

In a further embodiment, R⁴ is methyl or ethyl.

In a further embodiment, R⁵ is hydrogen or methyl.

In a further embodiment, R⁶ is methyl and R⁷ is hydrogen. In a further embodiment, R⁶ is hydrogen and R⁷ is hydrogen.

In a further embodiment, Z is

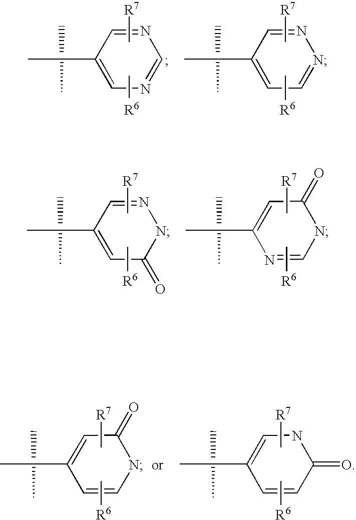

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

The compounds of the present invention are useful in therapy, in particular for the treatment of neurological, psychiatric, pain, and gastrointestinal disorders.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl, acetic acid or a methanesulfonic acid to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The general terms used in the definition of formula I have the following meanings:

Halogen as used herein is selected from chlorine, fluorine, bromine or iodine.

C₁-C₃ alkyl is a straight or branched allyl group, having from 1 to 3 carbon atoms, for example methyl, ethyl, n-propyl or isopropyl.

C₁-C₃ alkoxy is an alkoxy group having 1 to 3 carbon atoms, for example methoxy, ethoxy, isopropoxy or n-propoxy.

All chemical names were generated using ACDLABS v. 9.04 or 10.06.

In formula I above, X may be present in any of the two possible orientations.

### Pharmaceutical Composition

The compounds of the present invention may be formulated into conventional pharmaceutical compositions comprising a compound of formula I, or a pharmaceutically acceptable salt or solvate thereof in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely so divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10% w to 50% w, of a compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

The compounds according to the present invention are useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of formula I, as defined hereinbefore, for use in therapy.

The invention relates to compounds of formula I, as defined hereinbefore, for use in treatment of mGluR5-mediated disorders.

The invention relates to compounds of formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I, as defined above, for use in treatment of pain related to migraine) inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout.

The invention relates to compounds of formula I as defined hereinbefore, for use in treatment of stroke, head trauma, anoxic and ischemic injuries, hypoglycemia, cardiovascular diseases and epilepsy.

The present invention relates also to the use of a compound of formula I as defined hereinbefore, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates a compound of formula I for the inhibition of transient lower esophageal sphincter relaxations, for the treatment of GERD, for the prevention of gastroesophageal reflux, for the treatment of regurgitation, for treatment of asthma, for treatment of laryngitis, for treatment of lung disease, for the management of failure to thrive, for the treatment of irritable bowel syndrome (IBS) and for the treatment of functional dyspepsia (FD).

Another embodiment of the invention relates to the use of a compound of formula I for the manufacture of a medicament for inhibition of transient lower esophageal sphincter relaxations, for the treatment of GERD, for the prevention of gastroesophageal reflux, for the treatment regurgitation, for treatment of asthma, for treatment of laryngitis, for treatment of lung disease, for the management of failure to thrive, for the treatment of irritable bowel syndrome (IBS) and for the treatment of functional dyspepsia (FD).

Another embodiment of the present invention relates to the use of a compound of formula I for treatment of overactive bladder or urinary incontinence.

The wording “TLESR”, transient lower esophageal sphincter relaxations, is herein defined in accordance with Mittal, R. K., Holloway, R. H., Penagini, R., Blackshaw, L. A., Dent, J., 1995, Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000, Diagnosis of reflux disease. Baillière's Clin. Gastroenterol 14, pp. 759-774.

The compounds of formula I above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

One embodiment of the present invention is a combination of a compound of formula I and an acid secretion inhibiting agent. A “combination” according to the invention may be present as a “Fix combination” or as a “kit of pails combination”. A “fix combination” is defined as a combination wherein the (i) at least one acid secretion inhibiting agent; and (ii) at least one compound of formula I are present in one unit. A “kit of parts combination” is defined as a combination wherein (i) at least one acid secretion inhibiting agent; and (ii) at least one compound of formula I are present in more than one unit. The components of the “kit of pants combination” may be administered simultaneously, sequentially or separately. The molar ratio of the acid secretion inhibiting agent to the compound of formula I used according to the invention in within the range of from 1:100 to 100:1, such as from 1:50 to 50:1 or from 1:20 to 20:1 or from 1:10 to 10:1. The two drugs may be administered separately in the same ratio. Examples of acid secretion inhibiting agents are H2 blocking agents, such as cimetidine, ranitidine; as well as proton pump inhibitors such as pyridinylmethylsulfinyl benzimidazoles such as omeprazole, esomeprazole, lansoprazole, pantoprazole, rabeprazole or related substances such as leminoprazole.

### Non-Medical Use

In addition to their use in therapeutic medicine, the compounds of formula I, as well as salts and hydrates of such compounds, are useful as pharmacological tools in the development and standardisation of in vitro and in vivo test systems for the evaluation of the effects of inhibitors of mGluR related activity in laboratory animals such as cats, dogs, rabbits, monkeys, rats and mice, as pant of the search for new therapeutic agents.

### Methods of Preparation

Another aspect of the present invention provides processes for preparing compounds of formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Green's Protective Groups in Organic Synthesis”, 4^{th} Edition, P. G. M. Wuts, T. W. Green, Wiley-Interscience, New York, (2006). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations”, 2^{nd} Edition R. C. Larock, VHC Publishers, Inc. (1999). References and descriptions of other suitable reactions are described in textbooks of organic chemistry, for example, “Advanced Organic Chemistry: Reactions, Mechanisms, and Structure”, 6^{th} Edition, Michael B. Smith and Jerry March, McGraw Hill (2007) or, “Organic Synthesis”, 2^{nd} Edition, Michael B. Smith, McGraw Hill, (2001). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The term “reflux” shall mean, unless otherwise stated, in reference to an employed solvent a temperature at or above the boiling point of named solvent.

### ABBREVIATIONS

Boc tert-Butoxycarbonyl
DCM Dichloromethane
DEA N,N-Diisopropyl ethylamine
DIBAL-H Diisobutylaluminium hydride
DIC N,N′-Diisopropylcarbodiimide
DMAP N,N-Dimethyl-4-aminopyridine
DMF N,N-Dimethylformamide
DMSO Dimethylsulfoxide
EDCI N-[3-(dimethylamino)propyl]-N′-ethylcarbodiimide hydrochloride
EDC 1-Ethyl-3-(3-dimethylaminopropyl)carbodiimide
Et₂O Diethylether
EtOAc Ethyl acetate
EtOH Ethanol
EtI Iodoethane
Et Ethyl
Fmoc 9-Fluorenylmethyloxycarbonyl
h Hour(s)
HOBt N-Hydroxybenzotriazole
HBTU O-(Benzotriazol-1-yl)-N,N,N′,N′-tetramethyluronium hexafluorophosphate
HPFC High performance flash chromatography
HPLC High performance liquid chromatography
IPA Isopropylalcohol
LAM Lithium aluminium hydride
LCMS Liquid chromatography mass spectrometry
LDA Lithium diisopropylamide
LG Leaving Group
MeCN Acetonitrile
MeOH Methanol
min Minutes
Met Iodomethane
MeMgCl Methyl magnesium chloride
Me Methyl
MTBE Methyl tert-butyl ether
n-BuLi 1-Butyllithium
NaOAc Sodium acetate
NMR Nuclear magnetic resonance
NMP N-Methyl pyrrolidinone
o.n. Over night
PG Protective Group
RT, rt, r.t. Room temperature
TEA Triethylamine
THF Tetrahydrofuran
nBu normal Butyl
OMs Mesylate or methane sulfonate ester
OTs Tosylate, toluene sulfonate or 4-methylbenzene sulfonate ester
TBAF Tetrabutylammonium fluoride
TBDMSCl tert-Butyldimethylchlorosilane
t-BuLi tert-Butyllithium
TFA Trifluoroacetic acid
TMS Tetramethylsilane
pTsOH p-Toluenesulfonic acid
RP Reversed Phase
SPE Solid phase extraction (usually containing silica gel for mini-chromatography)
sat. Saturated

### Preparation of Intermediates

The intermediates provided in synthetic paths given below, are useful for further preparation of compounds of formula I. Other starting materials are either commercially available or can be prepared via methods described in the literature. The synthetic pathways described below are non-limiting examples of preparations that can be used. One of skill in the art would understand other pathways might be used.

General Syntheses of 1,2,4-oxadiazole Compounds of Formula I

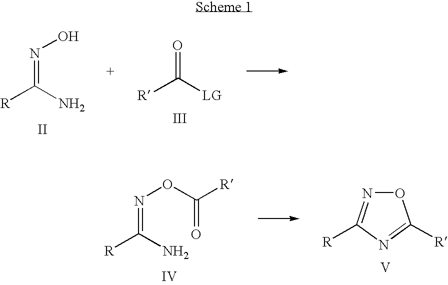

A compound of formula I, wherein X is a 1,2,4-oxadiazole (V) may be prepared through cyclization of a compound of formula IV, which in turn may be formed from a suitably activated compound of formula III with a compound of formula II.

Compounds of formula II may be prepared from a suitable nitrile, The compound of formula III may be activated in the following non-limiting ways: i) as the acid chloride formed from the acid using a suitable reagent such as oxalyl chloride or thionyl chloride; ii) as an anhydride or mixed anhydride formed from treatment with a reagent such as allyl chloroformate; iii) using traditional methods to activate acids in amide coupling reactions such as EDCI with HOBt or uronium salts like HBTU; iv) as an alkyl ester when the hydroxyamidine is deprotonated using a strong base like sodium tert-butoxide or sodium hydride in a solvent such as EtOH or toluene at elevated temperatures (50° C.-110° C.). The transformation of compounds II and III into compounds of type V may be performed as two consecutive steps via an isolated intermediate of type IV, as described above, or the cyclization of the intermediate formed in situ may occur spontaneously during the ester formation. The formation of ester IV may be accomplished using an appropriate aprotic solvent such as DCM, THF, DMF or toluene, with optionally an appropriate organic base such as TEA, DEA and the like or an inorganic base such sodium bicarbonate or potassium carbonate. The cyclization of compounds of formula IV to form an oxadiazole may be carried out on the crude ester with evaporation and replacement of the solvent with a higher boiling solvent such as DMF or with aqueous extraction to provide a semi-purified material or with material purified by standard chromatographic methods. The cyclization may be accomplished by heating conventionally or by microwave irradiation (100° C.-180° C.), in a suitable solvent such as pyridine or DMF or using a lower temperature method employing reagents like TBAF in THF or by any other suitable known literature method.

Further examples of the above described reactions can be found in Poulain et al., Tetrahedron Lett., (2001), 42, 1495-98, Ganglott et al., Tetrahedron Lett., (2001), 42, 1441-43, and Mathvink et al, Bioorg. Med. Chem. Lett. (1999), 9, 1869-74, which are hereby included as references.

### Synthesis of Aryl Nitriles and Carboxylic Acids for Use in Preparation of Compounds of Formula I

Aryl nitrites are available by a variety of methods including cyanation of an aryl halide or triflate via palladium or nickel catalysis using an appropriate cyanide source such as zinc cyanide in an appropriate solvent such as DMF. The corresponding carboxylic acid is available from the nitrile by hydrolysis under either acidic or basic conditions in an appropriate solvent such as aqueous alcohols. Aryl carboxylic acids are also available from a variety of other sources, including iodo- or bromo-lithium exchange followed by trapping with CO₂ to give directly the acid.

Carboxylic acids may be converted to primary amides using any compatible method to activate the acid, including via the acid chloride or mixed anhydride, followed by trapping with any source of ammonia, including ammonium chloride in the presence of a suitable base, ammonium hydroxide, ammonia in MeOH or ammonia in an aprotic solvent such as dioxane. This primary amide may be converted to the nitrile using a variety of dehydration reagents such as oxalyl chloride or thionyl chloride. The reaction sequence to convert an acid into a nitrile may also be applied to non-aromatic acids, including suitably protected amino acid derivatives. A suitable protecting group for an amine, in an amino acid or in a remote position of any other acid starting material, may be any group which removes the basicity and nucleophilicity of the amine functionality, including such carbamate protecting group as Boc.

Some acids are readily prepared taking advantage of commercially available analogs. For example, 6-methylpyridine-4-carboxylic acid is prepared by dechlorination of 2-chloro-6-methylpyridine-4-carboxylic acid. Certain types of substituted fluoro-benzonitriles and benzoic acids are available from bromo-difluoro-benzene via displacement of one fluoro group with a suitable nucleophile such as imidazole in the presence of a base such as potassium carbonate in a compatible solvent such as DMF at elevated temperatures (80-120° C.) for extended periods of time. The bromo group may subsequently be elaborated into the acid or nitrite as described above.

1,3-Disubstituted and 1,3,5-trisubstituted benzoic acids and benzonitriles may be prepared by taking advantage of readily available substituted isophthalic acid derivatives. Monohydrolysis of the diester allows selective reaction of the acid with a variety of reagents, most typically activating agents such as thionyl chloride, oxalyl chloride or isobutyl chloroformate and the like. From the activated acid, a number of products are available. In addition to the primary amide used to form the nitrite by dehydration as mentioned above, reduction to the hydroxymethyl analog may be carried out on the mixed anhydride or acid chloride using a variety of reducing agents such as sodium borohydride in a compatible solvent such as THF. The hydroxymethyl derivative may be further reduced to the methyl analog using catalytic hydrogenation with an appropriate source of catalyst such as palladium on carbon in an appropriate solvent such as EtOH. The hydroxymethyl group may also be used in any reaction suitable for benzylic alcohols such as acylation, alkylation, transformation to halogen and the like. Halomethylbenzoic acids of this type may also be obtained from bromination of the methyl derivative when not commercially available. Ethers obtained by alkylation of the hydroxymethyl derivatives may also be obtained from the halomethylaryl benzoate derivatives by reaction with the appropriate alcohol using an appropriate base such as potassium carbonate or sodium hydroxide in an appropriate solvent such as THF or the alcohol. When other substituents are present, these may also be employed in standard transformation reactions. E.g., treatment of anilines with acid and sodium nitrite may yield a diazonium salt which may be transformed into a halide such as fluoride using tetrafluoroboric acid. Phenols react in the presence of a suitable base such as potassium carbonate with alkylating agents to form aromatic ethers.

### Formation of Isoxazole Precursor of Compounds of Formula I

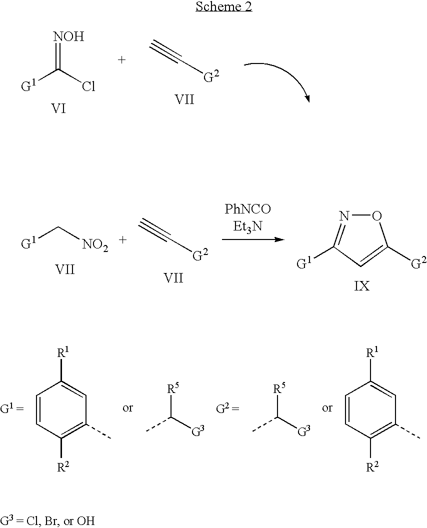

A compound of formula IX, wherein G¹ and/or G² is a moiety from an intermediate or group(s) as defined by formula I may be prepared by a 1,3-dipolar cycloaddition between compounds of formula VI and VII under basic conditions using a suitable base such as sodium bicarbonate or TEA at suitable temperatures (0° C.-100° C.) in solvents such as toluene. Synthesis of compounds of type VI has previously been described in the literature, e.g. Kim, Jae Nyoung; Ryu, Eung K; J. Org. Chem. (1992), 57, 6649-50. 1,3-Dipolar cycloaddition with acetylenes of type VII can also be effected using substituted nitromethanes of type VIII via activation with an electrophilic reagent such as PhNCO in the presence of a base such as TEA at elevated temperatures (50° C.-100° C.). Li, C-S.; Lacasse, E.; Tetrahedron Lett., (2002) 43; 3565-3568. Several compounds of type VII are commercially available, or may be synthesized by standard methods as known by one skilled in the art.

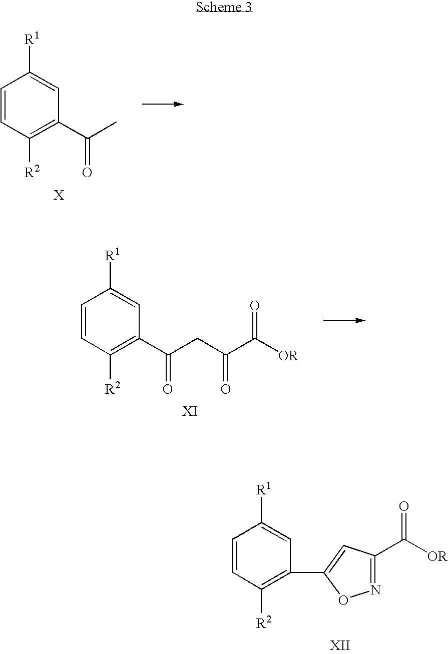

Alternatively, compounds of formula I, which are available from a Claisen condensation of a methyl ketone X and an ester using basic conditions (see scheme 3) using such bases as sodium hydride or potassium tert-butoxide, may yield compounds of formula XI via condensation and subsequent cyclization using hydroxylamine, for example in the form of the hydrochloric acid salt, at elevated temperatures (60° C.-120° C.) to afford intermediate XII.

It is understood that for both methods, subsequent functional group transformations of intermediates such as IX and XII may be necessary. In the case of an ester group as in XII, these transformations may include, but is not limited to either of the following three procedures: a) Complete reduction using a suitable reducing agent such as LAH in solvents such as THF. b) Partial reduction using a suitable selective reducing agent such as DIBAL-H followed by addition of an alkylmetal reagent. c) Addition of an alkylmetal reagent such as an allyl magnesium halide in solvents such as toluene or THF, followed by reduction with for example sodium borohydride in MeOH.

### Formation of Tetrazole Precursors of Compounds of Formula I

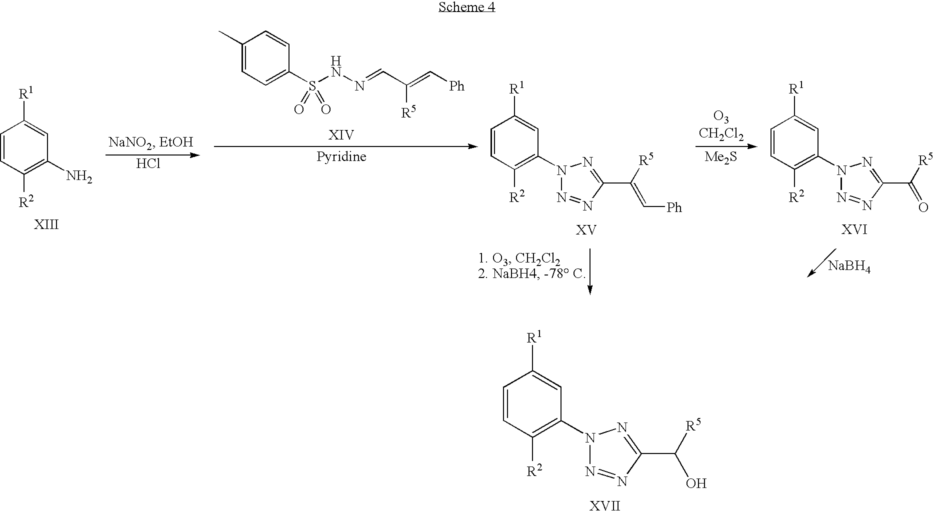

Compounds of formula I wherein X is tetrazole, as in intermediates XVI are prepared through condensation between arylsulphonylhydrazones XIV with diazonium salts derived from anilines XIII (scheme 4). The tetrazole intermediate XV, obtained from the diazonium salt of XIII and the arylsulphonylhydrazones of cinnamaldehydes can be cleaved to provide an aldehyde or ketone XV directly in a one-pot process using a reagent such as ozone or via the diol using a dihydroxylation reagent such as osmium tetroxide followed by subsequent cleavage using a reagent such as lead (IV) acetate; J. Med. Chem. (2000), 43, 953-970.

The olefin can also be converted in one pot to the alcohol via ozonolysis followed by reduction with a reducing agent such as sodium borohydride. Aldehydes XVI may be reduced to primary alcohols of formula XVII using well known reducing agents such as sodium or lithium borohydride, in a solvent such as MeOH, TIM or DMF at temperatures between 0° C.-80° C. Secondary alcohols may also be formed from aldehydes of formula XVI via addition reactions of an organometallic reagent, for example Grignard reagents (e.g. MeMgX), in a solvent such as THF at temperatures between −78° C. to 80° C., and are typically performed between 0° C. and room temperature.

Preparation of Amino[1,2,4]triazoles

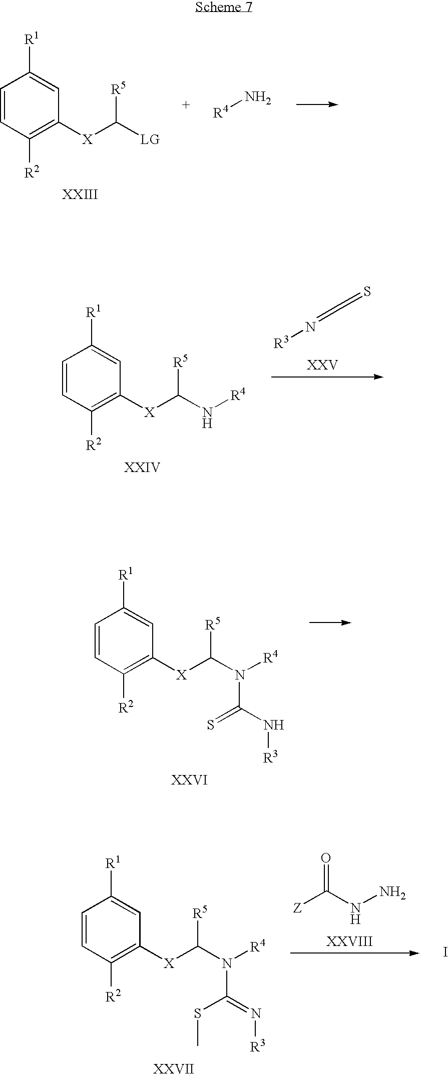

With reference to scheme 7, intermediates XXIII are obtained from the corresponding alcohol (LG=O) intermediates by standard methods to the corresponding halides (e.g. LG=Cl, Br etc.) by the use of for example triphenylphosphine in combination with either iodine, N-bromosuccinimide or N-chlorosuccinimide, or alternatively by treatment with phosphorous tribromide or thionyl chloride. In a similar fashion alcohols may be transformed to other LC such as mesylates or tosylates by employing the appropriate sulfonyl halide or sulfonyl anhydride in the presence of a non-nucleophilic base together with the alcohol to obtain the corresponding sulfonates. Alkyl chlorides or sulphonates can be converted to the corresponding bromides. The amines XXIV are made from XXII by reaction with the amine in a solvent such as THF, NMP or DMF at temperatures from 0° C. to 60° C. The amines are reacted with a alkylisothiocyanate XXV to form XXVI in DCM, THF, NMP or DMF at −100 to 100° C. Isothioureas XXVII are obtained by S-allylation of the corresponding thioureas with an alkylhalide, e.g. MeI or EtI in acetone, EtOH, THF, DCM or the like at −100 to 100° C. The final step in synthesis of compounds of formula I involves reaction between XXVII and an acylhydrazid in a solvent such as DMSO, IPA, EtOH or DMF at 0° C.-180° C.

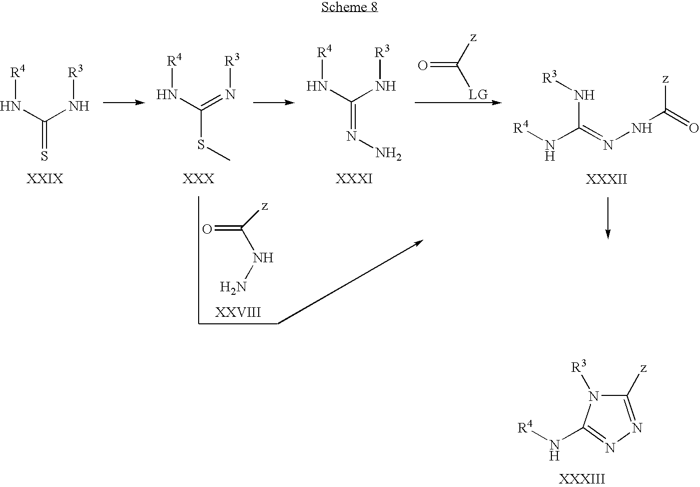

Amino[1,2,4]triazoles XXXIII (scheme 8, R groups defined as in formula I) are obtained by treating carbonohydrazonic diamides XXXI with a proper acylating agent carrying a LG in suitable solvent such as TH-F, pyridine or DMF at −20 to 100° C. The reaction initially leads to an open intermediate XXXII that either forms a triazole ring spontaneously, or can be made to do so by heating at 50 to 200° C. in for example pyridine or DMF. The LG may be chloro or any other suitable LG generated in situ by treatment of the corresponding carboxylic acid (LG is OH) with standard activating reagents as described herein below. Carbonohydrazonic diamides XXXI may be generated from isothioureas XXX, in which the S-alkyl (for example S-Me as shown in scheme 5) moiety acts as a leaving group upon treatment with hydrazine in solvents such as pyridine, MeOH, EtOH, IPA, THF, DMSO or the like at −20 to 100° C. The open intermediate XXXII can also be directly generated by treatment of isothioureas with acylhydrazines under the same conditions as described for the reaction with hydrazine. Isothioureas are obtained by S-alkylation of the corresponding thioureas with for example MeI or EtI in acetone, EtOH, THF, DCM or the like at −100 to 100° C.

Compounds of formula I can be prepared from XXXIII by bond formation through nucleophilic replacement of a leaving group (LG) in which the aminomethyl triazole NH moiety is acting as nucleophile. The said nitrogen atom of the aminomethyl triazole in its anionic form, generated by treatment of the corresponding protonated neutral atom with bases in suitable solvents such as LDA or nBuLi in THF, diethyl ether or toluene, or sodium hydride or NaOtBu in for example DMF or DMSO, or K₂CO₃ in acetonitrile or ketones such as 2-butanone at a temperature from −100 to 150° C. The LG is preferably chloro, bromo, OMs and OTs.

The acylhydrazines of formula XXVIII are commercially available or can be synthesised from the corresponding alkyl esters by heating with hydrazine in a solvent such as MeOH, EtOH or THF at a temperature from rt to 100° C.

### Synthesis of 1,2,3-Triazoles

Alkyne XXXIV, PG=protective group, may be transformed into XXXVI e.g. by treatment with a halogenated substituted phenyl of formula XXXV (scheme 5 wherein LG=1) with sodium azide and a copper-catalyst in a solvents mixture like DMSO/H₂O at 20° C.-100° C. (see J. Org. Chem. 2002, 67, 3057).

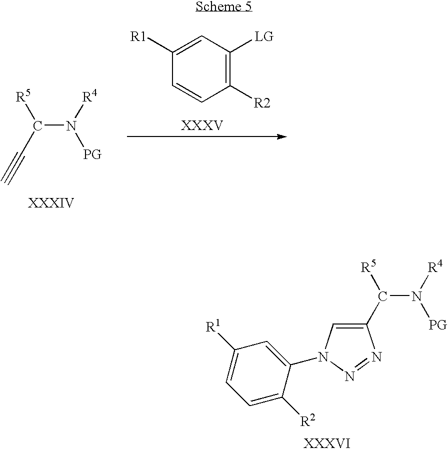

An alternative regioisomer such as XXXVIII, scheme 6, may be synthesized either from a substituted triazole XXXVII which may undergo a nucleophilic addition to a halogenated phenyl such as XXXV (scheme 6, LG=F), using an inorganic base such as K₂CO₃ in DMSO, (Tetrahedron, (2001), 57 (22), 4781-4785), or from an α-hydroxyketone XXXIX which may be reacted with an aryl hydrazine, XL, in the presence of e.g. cupric chloride and heating, (Synth. Commun., (2006), 36, 2461-2468).

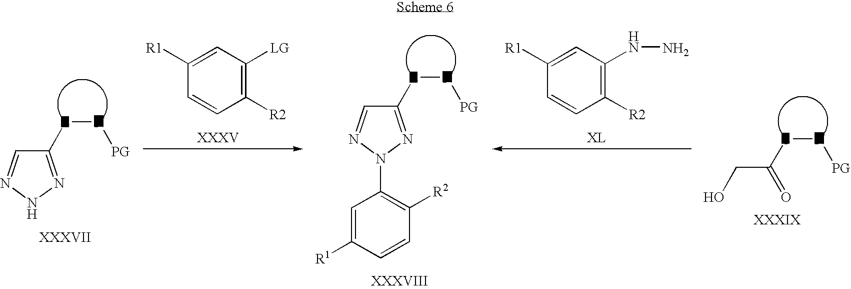

### EXAMPLES

The invention will now be illustrated by the following non-limiting examples.

### General Methods

All starting materials are commercially available or earlier described in the literature. The ¹H and ¹³C NMR spectra were recorded either on Varian Mercery Plus or Varian INOVA spectrometers operating at 300, 400 and 600 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was 13 kV and the mass spectrometer was scanned from m/z 100-700 at a scan time of 0.8 s. To the column, SunFire C18 2.5μ 3×20 mm was applied a linear gradient from 5% to 100% MeCN in a pH 3: formate buffer or a pH 7: acetate buffer.

Preparative reversed phase chromatography was run on a Waters Delta Prep Systems with a diode array detector using an Kromasil C8, 10 μm columns. Purification of products were also done by flash chromatography in silica-filled glass columns. Microwave heating was performed in a Smith Synthesizer Single-mode microwave cavity producing continuous irradiation at 2450 MHz (Personal Chemistry AB, Uppsala, Sweden).

### Example 1.1

### N-{[5-(3-Chlorophenyl)isoxazol-3-yl]methyl}cyclopropanamine

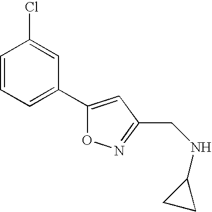

[5-(3-Chlorophenyl)isoxazol-3-yl]m ethyl methanesulfonate (WO 2004/014881) (1.45 g, 5.04 mmol) was dissolved in THF (30 mL) and cyclopropylamine (2.0 mL, 25 mmol) was added. The reaction mixture was stirred at it overnight. The next day, more cyclopropyl-amine (2.0 mL, 25 mmol) was added and the reaction mixture was heated at 40° C. with reflux condenser for 3 h. The solvent was evaporated and the residue was dissolved in DCM (80 mL), washed with saturated NaHCO₃ solution (50 mL) and dried (MgSO₄). The crude title compound (yield 90%) was used in the next step without further purification.

¹H NMR (400 MHz, CDCl₃): δ 7.75 (d, 1H), 7.65 (m, 1H), 7.40 (m, 2H), 6.56 (s, 1H), 4.76 (br s, 1H), 3.97 (s, 3H), 2.26 (m, 1H), 0.50 (m, 2H), 0.44 (m, 2H).

In a similar manner were the following compounds synthesised

| Example | Structure | Name | Yield |
|---|---|---|---|
| 1.2 | 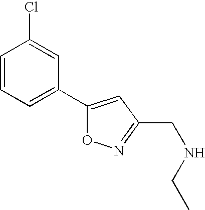 | N-{[5-(3-Chlorophenyl)isoxazol-3-yl]methyl}ethanamine | 75%0.75 g |
| ¹H NMR | (400 MHz, DMSO-d6): δ 7.89 (m, 1H), 7.78 (m, 1H), 7.52 (m, 2H), 7.07 (s, 1H), 3.71(s, 2H), 2.51 (q, 2H), 0.99 (t, 3H) | | |
| 1.3 | 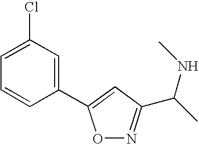 | 1-[5-(3-Chlorophenyl)isoxazol-3-yl]-N-methylethanamine | 97% 1.1 g |
| ¹H NMR | (400 MHz, CDCl₃): δ 7.39 (s, 1H), 7.63 (m, 1H), 7.37 (m, 2H), 7.51 (s, 1H), 3.92 (q, | | |
| | 1H), 2.38 (s, 3H), 1.43 (d, 3H) | | |
| 1.4 | 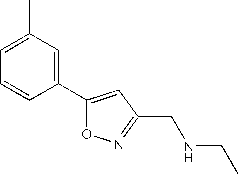 | N-{[5-(3-Methylphenyl)isoxazol-3-yl]methyl}ethanamine | Quant.0.25 g |
| ¹H NMR | (400 MHz, CDCl₃): δ 7.55 (m, 2H), 7.31 (t, 1H), 7.21 (d, 1H), 6.49 (s, 1H), 3.88 (s, | | |
| | 2H), 2.71 (q, 2H), 2.38 (s, 3H), 1.12 (t, 3H) | | |
| 1.5 | 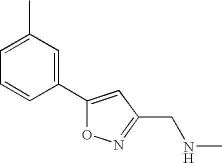 | N-Methyl-1-[5-(3-methylphenyl)isoxazol-3-yl]methanamine | Quant.0.39 g |
| ¹H NMR | (400 MHz, CDCl₃): δ 7.62 (s, 1H), 7.59 (d, 1H), 7.36 (t, 1H), 7.27 (m, 1H), 6.53 (s, | | |
| | 1H), 3.89 (s, 2H), 2.53 (s, 3H), 2.43 (s, 3H) | | |
| 1.6 | 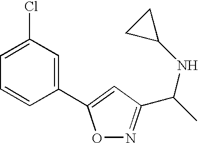 | N-{1-[5-(3-Chlorophenyl)isoxazol-3-yl]ethyl}cyclopropanamine | Quant0.27 g |
| ¹H NMR | (400 MHz, CDCl₃): δ 7.75 (m, 1H), 7.65 (m, 1H), 7.39 (m, 2H), 6.51 (s, 1H), 4.13 (q, | | |
| | 1H), 2.13 (m, 1H), 1.45 (d, 3H), 0.38 (m, 4H) | | |
| 1.7 | 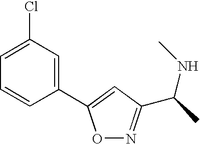 | (1S)-N-Methyl-1-[5-(3-methylphenyl)-isoxazol-3-yl]ethanamine | Quant 3.3 g |

| | |
|---|---|
| ¹H NMR | (400 MHz, DMSO-d6): δ 7.64 (m, 2H), 7.41 (m, 1H), 7.30 (m, 1H), 6.94 (s, 1H), 3.75 |
| | (q, 1H), 2.37 (s, 3H), 2.19 (s, 3H), 1.33 (d, 3H) |

### Example 2.1

### 1-{[5-(3-Chlorophenyl)isoxazol-3-yl]methyl}-1-cyclopropyl-3-methylthiourea

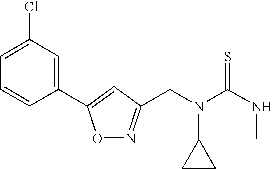

The title compound of Example 1.1 (1.1 g, 4.43 mmol) was dissolved in (DCM, 25 mL) and methyl isothiocyanate (0.5 g, 6.8 mmol) was added. The reaction mixture was stirred at rt over night, washed with water (2×20 mL) and dried (MgSO₄). The product precipitated from MeCN/water and was filtered and dried under reduced pressure to give the title compound (0.80 g, 56%).

¹H NMR (400 MHz, CDCl₃): δ 7.72 (s, 1H), 7.62 (m, 1H), 7.36 (m, 2H), 6.75 (s, 1H), 6.68 (br s, 1H), 5.30 (s, 2H), 3.21 (d, 3H), 2.53 (m, 1H), 0.96 (m, 2H), 0.90 (m, 2H).

In a similar manner were the following compounds synthesised:

| Example | Structure | Name | Yield |
|---|---|---|---|
| 2.2 | 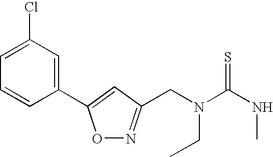 | 1-{[5-(3-Chlorophenyl)isoxazol-3-yl]methyl}-1-ethyl-3-methylthiourea | 55%0.53 g |
| ¹H NMR | (400 MHz, CDCl₃): δ 7.73 (s, 1H), 7.62 (m, 1H), 7.37 (m, 2H), 6.78 (s, 1H), 5.76 (br d, | | |
| | 1H), 5.12 (s, 2H), 3.58 (q, 2H), 3.17 (d, 3H), 1.21 (t, 3H) | | |
| 2.3 | 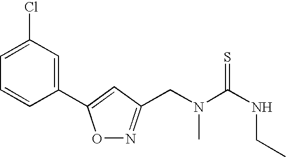 | 1-{[5-(3-Chlorophenyl)isoxazol-3-yl]methyl}-3-ethyl-1-methylthiourea | 85% 1.2 g |
| ¹H NMR | (400 MHz, CDCl₃): δ 7.73 (s, 1H), 7.62 (m, 1H), 7.36 (m, 2H), 6.74 (s, 1H), 5.54 (br s, | | |
| | 1H), 5.22 (s, 2H), 3.69 (m, 2H), 3.11 (s, 3H), 1.22 (t, 3H) | | |
| 2.4 | 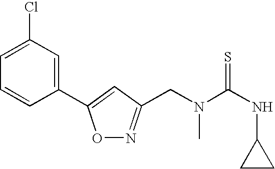 | 1-{[5-(3-Chlorophenyl)isoxazol-3-yl]methyl}-3-cyclopropyl-1-methylthiourea | 84% 1.3 g |
| ¹H NMR | (400 MHz, CDCl₃): δ 7.74 (m, 1H), 7.63 (m, 1H), 7.38 (m, 2H), 6.75 (s, 1H), 5.77 (br s, | | |
| | 1H), 5.20 (s, 2H), 3.08 (m, 4H), 0.96 (m, 2H), 0.61 (m, 2H) | | |
| 2.5 | 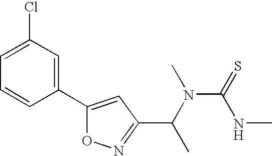 | 1-{1-[5-(3-Chlorophenyl)isoxazol-3-yl]ethyl}-1,3-dimethylthiourea | 69% 1.0 g |
| ¹H NMR | (400 MHz, CDCl₃): δ 7.72 (s, 1H), 7.61 (m, 1H), 7.37 (m, 2H), 7.06 (q, 1H), 6.55 (s, | | |
| | 1H), 5.62 (s, 1H), 3.21 (d, 3H), 2.87 (s, 3H), 1.61 (d, 3H) | | |
| 2.6 | 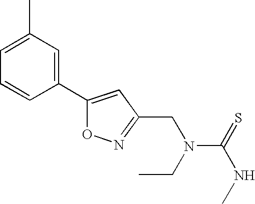 | 1-Ethyl-3-methyl-1-{[5-(3-methylphenyl)isoxazol-3-yl]methyl}thiourea | 92%0.29 g |
| ¹H NMR | (400 MHz, CDCl₃): δ 7.55 (m, 2H), 7.31 (t, 1H), 7.21 (d, 1H), 6.69 (s, 1H), 5.79 (m, | | |
| | 1H), 5.07 (s, 2H), 3.62 (q, 2H), 3.11 (d, 3H), 2.38 (s, 3H), 1.21 (t, 3H) | | |
| 2.7 | 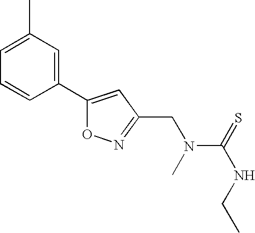 | 3-Ethyl-1-methyl-1-{[5-(3-methylphenyl)isoxazol-3-yl]methyl}thiourea | 76%0.22 g |
| ¹H NMR | (400 MHz, CDCl₃): δ 7.54 (m, 2H), 7.30 (t, 1H), 7.21 (d, 1H), 6.67 (s, 1H), 5.55 (m, | | |
| | 1H), 5.19 (s, 2H), 3.68 (m, 2H), 3.12 (s, 3H), 2.37 (s, 3H), 1.23 (t, 3H) | | |
| 2.8 | 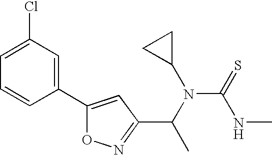 | 1-{1-[5-(3-Chlorophenyl)isoxazol-3-yl]ethyl}-1-cyclopropyl-3-methylthiourea | 73%0.25 g |
| ¹H NMR | (400 MHz, CDCl₃): δ 7.73 (m, 1H), 7.64 (m, 1H), 7.38 (m, 2H), 6.95 (q, 1H), 6.73 (m, | | |
| | 1H), 6.61 (s, 1H), 3.27 (d, 3H), 2.45 (m, 1H), 1.83 (d, 3H), 0.86 (m, 3H), 0.62 (m, 1H) | | |
| 2.9 | 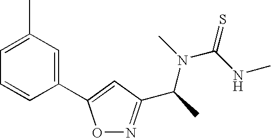 | 1,3-Dimethyl-1-{(1S)-1-[5-(3-methylphenyl)isoxazol-3-yl]ethyl}thiourea | 74% 3.2 g |

| | |
|---|---|
| ¹H NMR | (400 MHz, CDCl₃): δ 7.55 (m, 2H), 7.33 (m, 1H), 7.24 (m, 1H), 7.05 (q, 1H), 6.51 (s, |
| | 1H), 5.66 (m, 1H), 3.23 (d, 3H), 2.90 (s, 3H), 2.40 (s, 3H), 1.63 (d, 3H) |

### Example 3.1

### Methyl N-{[5-(3-chlorophenyl)isoxazol-3-yl]methyl}-N-cyclopropyl-N′-methylimidothiocarbamate

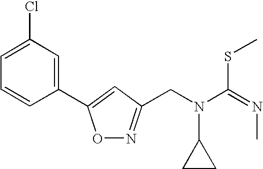

the title compound of Example 2.1 (0.80 g, 2.47 mmol) was dissolved in THF (20 mL) and the solution was cooled with an ice bath. NaOtBu (0.30 g, 3.12 mmol) and MeI (0.28 mL, 4.5 mmol) were added and the reaction mixture was stirred at 0° C. for 4 h. The solvent was evaporated in vacuo and the residue was partitioned between DCM (50 mL) and water (50 mL) and the organic layer was dried (MgSO₄) to give the crude title compound after removal of solvents in vacuo (0.76 g, 92%). The isolated material was used in the next step without further purification.

¹H NMR (400 MHz, CDCl₃): δ 7.71 (m, 1H), 7.62 (m, 1H), 7.37 (m, 2H), 6.45 (s, 1H), 4.63 (s, 2H), 3.27 (s, 3H), 2.56 (m, 1H), 2.30 (s, 3H), 0.75 (m, 2H), 0.55 (m, 2H).

In a similar manner the following compounds were synthesised:

| Example | Structure | Name | Yield |
|---|---|---|---|
| 3.2 | 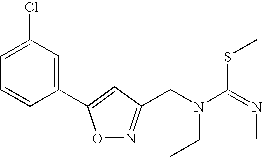 | Methyl N-{[5-(3-chlorophenyl)isoxazol-3-yl]methyl}-N-ethyl-N′-methylimidothiocarbamate | 92%0.50 g |
| ¹H NMR | (400 MHz, CDCl₃): δ 7.72 (s, 1H), 7.62 (m, 1H), 7.37 (m, 2H), 6.48 (s, 1H), 4.62 (s, 2H), | | |
| | 3.38 (q, 2H), 3.26 (s, 3H), 2.31 (s, 3H), 1.10 (t, 3H) | | |
| 3.3 | 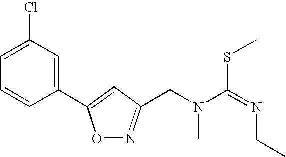 | Methyl N-{[5-(3-chlorophenyl)isoxazol-3-yl]methyl}-N′-ethyl-N-methylimidothiocarbamate | 84% 1.0 g |
| ¹H NMR | (400 MHz, CDCl₃): δ 7.70 (s, 1H), 7.60 (m, 1H), 7.36 (m, 2H), 6.48 (s, 1H), 4.62 (s, 2H), | | |
| | 3.52 (q, 2H), 2.90 (s, 3H), 2.31 (s, 3H), 1.13 (t, 3H) | | |
| 3.4 | 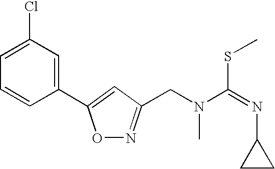 | Methyl N-{[5-(3-chlorophenyl)isoxazol-3-yl]methyl}-N′-cyclopropyl-N-methylimidothiocarbamate | 94% 1.3 g |
| ¹H NMR | (400 MHz, CDCl₃): δ 7.71 (s, 1H), 7.61 (m, 1H), 7.37 (m, 2H), 6.45 (s, 1H), 4.59 (s, 2H), | | |
| | 3.20 (m, 1H), 2.89 (s, 3H), 2.37 (s, 3H), 0.72 (m, 2H), 0.64 (m, 2H) | | |
| 3.5 | 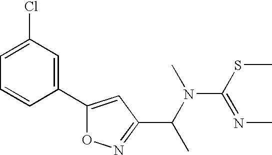 | Methyl N-{1-[5-(3-chlorophenyl)isoxazol-3-yl]ethyl}-N,N′-dimethylimidothiocarbamate | 100% 1.3 g |
| ¹H NMR | (400 MHz, CDCl₃): δ 7.70 (s, 1H), 7.61 (m, 1H), 7.36 (m, 2H), 6.43 (s, 1H), 5.66 (q, 1H), | | |
| | 3.27 (s, 3H), 2.74 (s, 3H), 2.33 (s, 3H), 1.59 (d, 3H) | | |
| 3.6 | 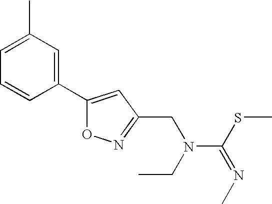 | Methyl N-ethyl-N′-methyl-N-{[5-(3-methylphenyl)isoxazol-3-yl]methyl}-imidothiocarbamate | 86%0.25 g |
| ¹H NMR | (400 MHz, CDCl₃): δ 7.53 (m, 2H), 7.30 (t, 1H), 7.20 (d, 1H), 6.40 (s, 1H), 4.60 (s, 2H), | | |
| | 3.39 (q, 2H), 3.26 (s, 3H), 2.38 (s, 3H), 2.31 (s, 3H), 1.10 (t, 3H) | | |
| 3.7 | 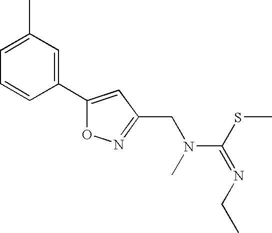 | Methyl N′-ethyl-N-methyl-N-{[5-(3-methylphenyl)isoxazol-3-yl]methyl}-imidothiocarbamate | 92%0.20 g |
| ¹H NMR | (400 MHz, CDCl₃): δ 7.52 (m, 2H), 7.30 (t, 1H), 7.20 (d, 1H), 6.42 (s, 1H), 4.61 (s, 2H), | | |
| | 3.52 (q, 2H), 2.89 (s, 3H), 2.37 (s, 3H), 2.31 (s, 3H), 1.14 (t, 3H) | | |
| 3.8 | 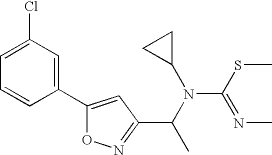 | Methyl N-{1-[5-(3-chlorophenyl)isoxazol-3-yl]ethyl}-N-cyclopropyl-N′-methylimidothiocarbamate | 99%0.23 g |
| ¹H NMR | (400 MHz, CDCl₃): δ 7.66 (m, 1H), 7.57 (m, 1H), 7.31 (m, 2H), 6.46 (s, 1H), 5.20 (q, 1H), | | |
| | 3.21 (s, 3H), 2.33 (m, 1H), 2.29 (s, 3H), 1.67 (d, 3H), 0.70 (m, 1H), 0.51 (m, 2H), 0.20 (m, | | |
| | 1H) | | |
| 3.9 | 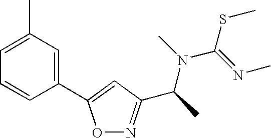 | Methyl N,N′-dimethyl-N-{(1S)-1-[5-(3-methylphenyl)isoxazol-3-yl]ethyl}imidothiocarbamate | 98% 3.3 g |

| | |
|---|---|
| ¹H NMR | (400 MHz, CDCl₃): δ 7.56 (m, 2H), 7.33 (m, 1H), 7.24 (m, 1H), 6.40 (s, 1H), 5.69 (m, |
| | 1H), 3.30 (s, 3H), 2.75 (s, 3H), 2.40 (s, 3H), 2.36 (s, 3H), 1.61 (d, 3H) |

### Example 4.1

### 5-{5-[{[5-(3-Chlorophenyl)isoxazol-3-yl]methyl}(cyclopropyl)amino]-4-methyl-4H-1,2,4-triazol-3-yl}pyridazin-3(2H)-one

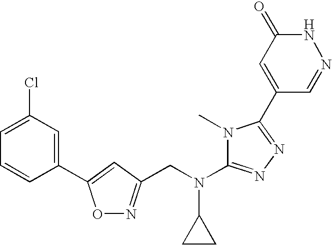

The title compound of Example 3.1 (0.25 g, 0.74 mmol) and the title compound of Example 10 (0.14 g, 0.89 mmol) were added to DMSO (3.0 mL) and the reaction mixture was heated to 120° C. for 1.5 h and purified on RP-HPLC with a gradient MeCN (10-50%) in a buffer of 0.2% AcOH in water:MeCN 95:5 to give the title compound (0.20 g, 63%).

¹H NMR (400 M-Hz, CDCl₃): δ 11.6 (br s, 1H), 8.49 (d, 1H), 7.71 (m, 1H), 7.60 (m, 1H), 7.37 (m, 2H), 7.07 (d, 1H), 6.59 (s, 1H), 4.63 (s, 2H), 3.71 (s, 3H), 3.00 (m, 1H), 0.80 (m, 2H), 0.64 (m, 2H).

The following compounds were synthesised in a similar manner:

| Example | Structure | Name | Yield |
|---|---|---|---|
| 4.2 | 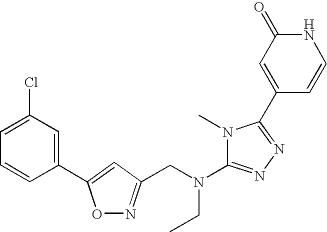 | 4-{5-[{[5-(3-Chlorophenyl)-isoxazol-3-yl]methyl}(ethyl)amino]-4-methyl-4H-1,2,4-triazol-3-yl}pyridin-2(1H)-one | 26%0.032 g |
| ¹H NMR | (400 MHz, CDCl₃): δ 7.72 (s, 1H), 7.61 (m, 1H), 7.46 (d, 1H), 7.36 (m, 2H), 6.90 (d, 1H), 6.77 (m, 2H), | | |
| | 4.56 (s, 2H), 3.55 (s, 3H), 3.26 (q, 2H), 1.22 (t, 3H) | | |
| 4.3 | 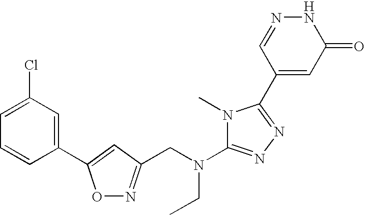 | 5-{5-[{[5-(3-Chlorophenyl)-isoxazol-3-yl]methyl}(ethyl)-amino]-4-methyl-4H-1,2,4-triazol-3-yl}pyridazin-3(2H)-one | 44%0.055 g |
| ¹H NMR | (400 MHz, CDCl₃): δ 11.06 (br s, 1H), 8.50 (m, 1H), 7.72 (s, 1H), 7.62 (m, 1H), 7.37 (m, 2H), 7.06 (m, | | |
| | 1H), 6.74 (s, 1H), 4.58 (s, 2H), 3.67 (s, 3H), 3.28 (q, 2H), 1.24 (t, 3H) | | |
| 4.4 | 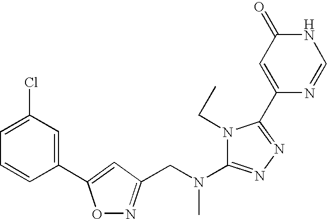 | 6-{5-[{[5-(3-chlorophenyl)-isoxazol-3-yl]methyl}(methyl)amino]-4-ethyl-4H-1,2,4-triazol-3-yl}pyrimidin-4(3H)-one | 22%0.051 g |
| ¹H NMR | (400 MHz, DMSO-d6): δ 12.88 (broad s, 1H), 8.31 (s, 1H), 7.94 (s, 1H), 7.80 (m, 1H), 7.53 (m, 2H), | | |
| | 7.15 (s, 1H), 6.83 (s, 1H), 4.39 (s, 2H), 4.32 (q, 2H), 2.84 (s, 3H), 1.23 (t, 3H) | | |
| 4.5 | 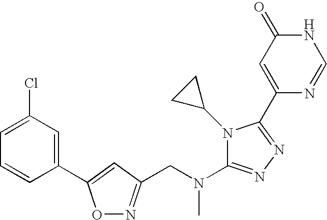 | 6-{5-[{[5-(3-chlorophenyl)isoxazol-3-yl]methyl}(methyl)amino]-4-cyclopropyl-4H-1,2,4-triazol-3-yl}pyrimidin-4(3H)-one | 15%0.038 g |
| ¹H NMR | (400 MHz, CDCl₃): δ 8.27 (s, 1H), 7.74 (m, 1H), 7.63 (m, 1H), 7.37 (m, 2H), 7.20 (s, 1H), 6.74 (s, 1H), | | |
| | 4.70 (s, 2H), 3.44 (m, 1H), 3.10 (s, 3H), 1.07 (m, 2H), 0.89 (m, 2H) | | |
| 4.6 | 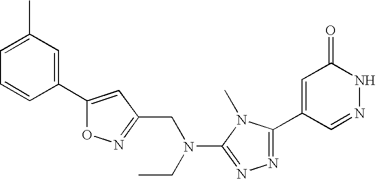 | 5-[5-(Ethyl{[5-(3-methylphenyl)isoxazol-3-yl]-methyl}amino)-4-methyl-4H-1,2,4-triazol-3-yl]pyridazin-3(2H)-one | 40% 0.13 g |
| ¹H NMR | (400 MHz, DMSO-d6): δ 13.22 (br s, 1H), 8.26 (d, 1H), 7.69 (s, 1H), 7.64 (d, 1H), 7.41 (t, 1H), 7.31 (d, | | |
| | 1H), 7.14 (d, 1H), 7.01 (s, 1H), 4.50 (s, 2H), 3.68 (s, 3H), 3.25 (q, 2H), 2.37 (s, 3H), 1.13 (t, 3H) | | |
| 4.7 | 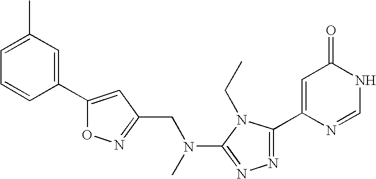 | 6-[4-Ethyl-5-(methyl{[5-(3-methylphenyl)isoxazol-3-yl]methyl}amino)-4H-1,2,4-triazol-3-yl]pyrimidin-4(3H)-one | 28%0.071 g |
| ¹H NMR | (400 MHz, DMSO-d6): δ 12.79 (br s, 1H), 8.30 (s, 1H), 7.64 (s, 1H), 7.60 (d, 1H), 7.36 (t, 1H), 7.26 (d, | | |
| | 1H), 6.95 (s, 1H), 6.84 (s, 1H), 4.37 (s, 2H), 4.31 (q, 2H), 2.82 (s, 3H), 2.33 (s, 3H), 1.21 (t, 3H) | | |
| 4.8 | 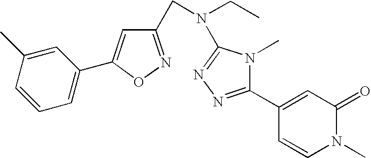 | 4-[5-(Ethyl{[5-(3-methylphenyl)isoxazol-3-yl]methyl}amino)-4-methyl-4H-1,2,4-triazol-3-yl]-1-methylpyridin-2(1H)-one | 594 mg64% |
| ¹H NMR | (500 MHz, CDCl₃): δ 7.59-7.55 (m, 2H), 7.39 (d, 1H), 7.33 (t, 1H), 7.25-7.22 (m, 1H), 6.85-6.82 (m, | | |
| | 1H), 6.76 (s, 1H), 6.70 (s, 1H), 4.56 (s, 2H), 3.65 (s, 3H), 3.60 (s, 3H), 3.28 (q, 2H), 2.40 (s, 3H), 1.23 (t, | | |
| | 3H) | | |
| 4.9 | 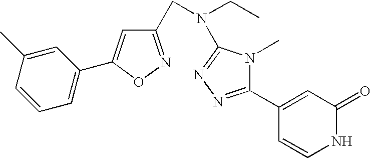 | 4-[5-(Ethyl{[5-(3-methylphenyl)isoxazol-3-yl]methyl}amino)-4-methyl-4H-1,2,4-triazol-3-yl]pyridin-2(1H)-one | 563 mg64% |
| ¹H NMR | (500 MHz, DMSO-d6): δ 11.76 (bs, 1H), 7.70-7.67 (m, 1H), 7.64 (d, 1H), 7.50 (d, 1H), 7.41 (t, 1H), 7.31 | | |
| | (d, 1H), 7.01 (s, 1H), 6.63 (s, 1H), 6.56 (d, 1H), 4.48 (s, 2H), 3.63 (s, 3H), 3.23 (q, 2H), 2.37 (s, 3H), | | |
| | 1.12 (t, 3H) | | |
| 4.10 | 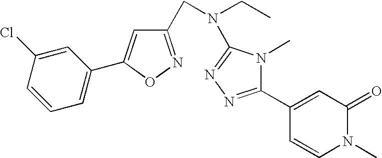 | 4-{5-[{[5-(3-Chlorophenyl)-isoxazol-3-yl]methyl}(ethyl)amino]-4-methyl-4H-1,2,4-triazol-3-yl}-1-methylpyridin-2(1H)-one | 37 mg29%Solid |
| ¹H NMR | (400 MHz, CDCl₃): δ 7.74-7.71 (m, 1H), 7.65-7.60 (m, 1H), 7.39-7.34 (m, 3H), 6.81 (dd, 1H), 6.75 (s, | | |
| | 1H), 6.73 (d, 1H), 4.55 (s, 2H), 3.62 (s, 3H), 3.57 (s, 3H), 3.24 (q, 2H), 1.21 (t, 3H) | | |
| 4.11 | 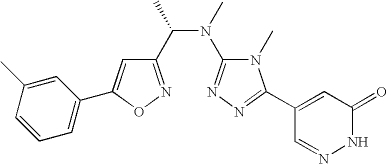 | (−)-5-[4-Methyl-5-(methyl-{(1S)-1-[5-(3-methylphenyl)isoxazol-3-yl]ethyl}amino)-4H-1,2,4-triazol-3-yl]pyridazin-3(2H)-one | 0.49 g52% |
| ¹H NMR | (400 MHz, DMSO-d6): δ 13.2 (bs, 1H), 8.27 (d, 1H), 7.68 (m, 2H), 7.43 (m, 1H), 7.32 (m, 1H), 7.13 (m, | | |
| | 2H), 4.84 (q, 1H), 3.70 (s, 3H), 2.76 (s, 3H), 2.38 (s, 3H), 1.60 (d, 3H). | | |
| | Optical rotation −199° (589 nm, MeCN, 0.5 g/100 mL, T 20° C.) | | |
| 4.12 | 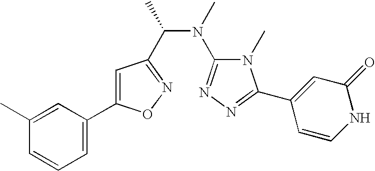 | (−)-4-[4-Methyl-5-(methyl-{(1S)-1-[5-(3-methylphenyl)-isoxazol-3-yl]ethyl}amino)-4H-1,2,4-triazol-3-yl]pyridin-2(1H)-one | 0.73 g75% |

| | |
|---|---|
| ¹H NMR | (400 MHz, CDCl₃): δ 7.59 (m, 2H), 7.46 (m, 1H), 7.34 (m, 1H), 7.25 (m, 1H), 6.96 (m, 1H), 6.81 (m, |
| | 1H), 6.76 (s, 1H), 4.89 (q, 1H), 3.68 (s, 3H), 2.87 (s, 3H), 2.41 (s, 3H), 1.73 (d, 3H). |
| | Optical rotation −181.0° (589 nm, MeCN, 1.1 g/100 mL, T 20° C.) |

### Example 5.1

### (−)-4-{5-[{1-[5-(3-Chlorophenyl)isoxazol-3-yl]ethyl}(methyl)amino]-4-methyl-4H-1,2,4-triazol-3-yl}-1-methylpyridin-2(1H)-one

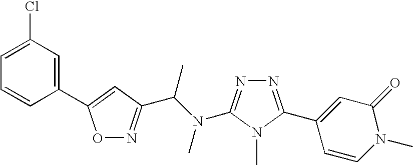

The title compound was synthesised according to the procedure for the title compound of Example 4.1 to give (132 mg, 53%). The racemic mixture was separated by chiral HPLC (ChiralcelOD—MeCN/TEA 100/0.1) and evaluated as single enantiomers, although the absolute configurations were not assigned.

¹H NMR (600 MHz, DMSO-d6): δ 7.95 (s, 1H), 7.81 (m, 2H), 7.54 (m, 2H), 7.28 (s, 1H), 6.67 (d, 1H), 6.57 (dd, 1H), 4.81 (q, 1H), 3.62 (s, 3H), 3.44 (s, 3H), 2.71 (s, 314, 1.55 (d, 3H). Optical rotation −163.3° (589 nm, MeCN, 1.0 g/100 mL, T 20° C.).

The following compound was synthesised in a similar manner. This racemic mixture was separated by chiral HPLC (ChiralcelOJ—Heptane/EtOH/TEA 60/40/0.1) and evaluated as single enantiomers, although the absolute configurations were not assigned:

| Example | Structure | Name | Yield |
|---|---|---|---|
| 5.2 | 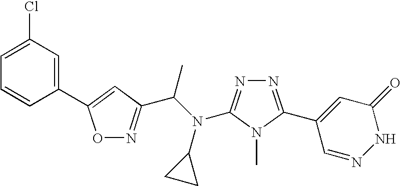 | 5-{5-[{1-[5-(3-Chlorophenyl)-isoxazol-3-yl]ethyl}(cyclopropyl)amino]-4-methyl-4H-1,2,4-triazol-3-yl}pyridazin-3(2H)-one | 0.038 g25% |

| | |
|---|---|
| ¹H NMR | (600 MHz, DMSO-d6): δ 13.2 (bs, 1H), 8.26 (d, 1H), 7.94 (m, 1H), 7.81 (m, 1H), 7.54 (m, 2H), 7.27 (s, |
| | 1H), 7.15 (m, 1H), 4.65 (q, 1H), 3.62 (s, 3H), 2.71 (m, 1H), 1.54 (d, 3H), 0.56 (m, 1H), 0.50 (m, 1H), 0.39 |
| | (m, 1H), 0.28 (m, 1H) |

### Example 6.1

### [5-(3-Methylphenyl)isoxazol-3-yl]methyl methanesulfonate

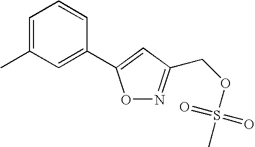

[5-(3-Methylphenyl)isoxazol-3-yl]methanol (1.92 g, 10.1 mmol) was dissolved in DCM (50 mL) and the reaction mixture was cooled to 0° C. and triethylamin (3.5 mL, 25.3 mmol) was added. Methanesulfonyl chloride (0.94 mL, 12.2 mmol) was added dropwise and the reaction mixture was stirred at 0° C. for 1 h and at it for 1 h. The reaction mixture was washed with saturated KHSO₄ solution (50 mL) and dried (MgSO₄) followed by removal of solvents in vacuo to give the title compound (2.55 g, 94%).

¹H NMR (400 MHz, CDCl₃): δ 7.57 (m, 2H), 7.34 (t, 1H), 7.25 (m, 1H), 6.63 (s, 1H), 5.32 (s, 2H), 3.07 (s, 3H), 2.40 (s, 3H).

The following compounds were synthesised in a similar manner:

| Example | Structure | Name | Yield |
|---|---|---|---|
| 6.2 | 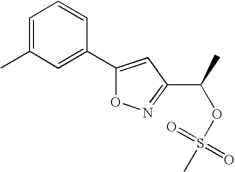 | (1R)-1-[5-(3-Methylphenyl)isoxazol-3-yl]ethyl methanesulfonate | 100%5.3 g |
| ¹H NMR | (400 MHz, DMSO-d6): δ 7.72 (s, 1H), 7.68 (m, 1H), 7.43 (m, 1H), 7.33 (m, | | |
| | 1H), 7.18 (s, 1H), 5.91 (q, 1H), 3.26 (s, 3H), 2.38 (s, 3H), 1.70 (d, 3H) | | |
| 6.3 | 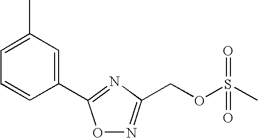 | [5-(3-Methylphenyl)-1,2,4-oxadiazol-3-yl]methylmethanesulfonate | 47%1.2 g |

| | |
|---|---|
| ¹H NMR | (400 MHz, DMSO-d6): δ 7.91-7.97 (m, 2H), 7.44 (m, 2H), 5.41 (s, 2H), 3.20 |
| | (s, 3H), 2.45 (s, 3H) |

### Example 7.1

### N,4-Dimethyl-5-pyrimidin-5-yl-4H-1,2,4-triazol-3-amine

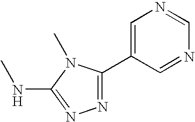

2-Amino-1,3-dimethyl-guanidine hydroiodide (1.1 g, 4.8 mmol) was dissolved in pyridine (30 mL) and cooled to −15° C. Pyrimidine-5-carbonyl chloride hydrochloride (0.86 g, 4.8 mmol) was added and the reaction mixture was stirred at −15° C. for 1 h, rt for 20 h and at 125° C. for 6 h. EtOH (100 mL) was added and the reaction mixture was stirred at it for 30 min. The solvents were evaporated in vacuo and the residue purified on RP-HPLC using a gradient of MeCN in 0.1 M NH₄OAc-buffer in water:MeCN 95:5 to give the title compound (0.18 g, 20%).

¹H NMR (400 MHz, D₂O): δ 9.13 (s, 1H), 8.92 (s, 2H), 3.33 (s, 3H), 2.82 (s, 3H).

The following compound was synthesised in a similar manner:

| Example | Structure | Name | Yield |
|---|---|---|---|
| 7.2 | 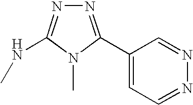 | N,4-Dimethyl-5-pyridazin-4-yl-4H-1,2,4-triazol-3-amine | 9%0.12 g |

| | |
|---|---|
| ¹H NMR | (400 MHz, MeOH-d4): δ 9.53 (m, 1H), 9.29 (m, 1H), 7.97 |
| | (dd, 1H), 3.55 (s, 3H), 2.98 (s, 3H) |

### Example 8.1

### N-{[5-(3-Chlorophenyl)isoxazol-3-yl]methyl}-N,4-dimethyl-5-pyridazin-4-yl-4H-1,2,4-triazol-3-amine

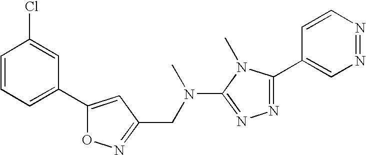

The title compound of Example 7.2 (46 mg, 0.24 mmol) was dissolved in DMF and NaH (19 mg, 60% dispersion in oil, 0.48 mmol) was added. [5-(3-chlorophenyl)isoxazol-3-yl]methyl methanesulfonate (WO 2004/014881) (70 mg, 0.24 mmol) was added and the reaction mixture was stirred at rt for 3 h. Purification by RP-HPLC with a gradient of 5-100% MeCN in 0.1 M NH₄OAc-buffer in water:MeCN 95:5 gave the title compound (73 mg, 79%).

¹H NMR (400 MHz, CDCl₃): δ 9.58 (s, 1H), 9.34 (d, 1H), 7.88 (dd, 1H), 7.74 (m, 1H), 7.64 (m, 1H), 7.39 (m, 2H), 6.77 (s, 1H), 4.60 (s, 2H), 3.75 (s, 3H), 3.03 (s, 3H).

The following compound was synthesised in a similar manner:

| Example | Structure | Name | Yield |
|---|---|---|---|
| 8.2 | 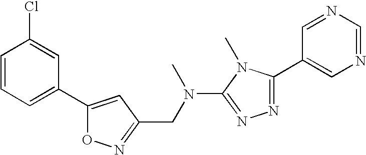 | N-{[5-(3-Chlorophenyl)isoxazol-3-yl]methyl}-N,4-dimethyl-5-pyrimidin-5-yl-4H-1,2,4-triazol-3-amine | 70%0.12 g |
| ¹H NMR | (400 MHz, CDCl₃): δ 9.32 (s, 1H), 9.12 (s, 2H), 7.74 (m, 1H), 7.64 (m, 1H), 7.39 (m, 2H), 6.77 (s, 1H), 4.64 | | |
| | (s, 2H), 3.68 (s, 3H), 3.04 (s, 3H) | | |
| 8.3 | 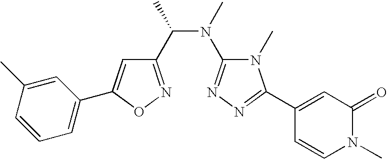 | 1-Methyl-4-[4-methyl-5-(methyl-{(1S)-1-[5-(3-methylphenyl)-isoxazol-3-yl]ethyl}amino)-4H-1,2,4-triazol-3-yl]pyridin-2(1H)-one | 33% 1.1 g |
| ¹H NMR | (600 MHz, DMSO-d6): δ 7.81 (m, 1H), 7.69 (s, 1H), 7.65 (m, 1H), 7.41 (m, 1H), 7.31 (m, 1H), 7.11 (s, 1H), | | |
| | 6.69 (s, 1H), 6.60 (m, 1H), 4.82 (q, 1H), 3.63 (s, 3H), 3.46 (s, 3H), 2.73 (s, 3H), 2.37 (s, 3H), 1.57 (d, 3H) | | |
| 8.4 | 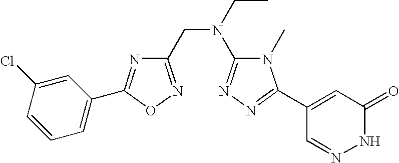 | 5-{5-[{[5-(3-Chlorophenyl)-1,2,4-oxadiazol-3-yl]methyl}(ethyl)amino]-4-methyl-4H-1,2,4-triazol-3-yl}pyridazin-3(2H)-one | 29% 0.3 g |
| ¹H NMR | (400 MHz, DMSO-d6): δ 10.78 (bs, 1H), 8.52 (d, 1H), 8.09 (m, 1H), 7.96-8.00 (m, 1H), 7.55-7.60 (m, 1H), | | |
| | 7.47 (m, 1H), 7.09 (d, 1H), 4.64 (s, 2H), 3.76 (s, 3H), 3.43 (q, 2H), 1.25 (t, 3H) | | |
| 8.5 | 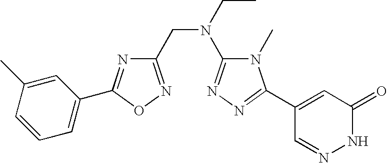 | 5-[5-(Ethyl{[5-(3-methylphenyl)-1,2,4-oxadiazol-3-yl]methyl}amino)-4-methyl-4H-1,2,4-triazol-3-yl]pyridazin-3(2H)-one | 51% 0.1 g |
| ¹H NMR | (400 MHz, DMSO-d6): δ 12.74 (bs, 1H), 8.52 (d, 1H), 7.89 (s, 1H), 7.87 (m, 1H), 7.39 (s, 1H), 7.37 (s, 1H), | | |
| | 7.14 (d, 1H), 4.61 (s, 2H), 3.77 (s, 3H), 3.42 (q, 2H), 2.41 (s, 3H), 1.23 (t, 3H) | | |
| 8.6 | 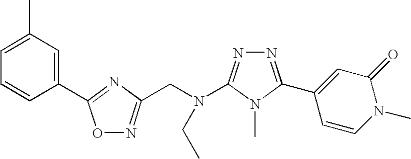 | 4-[5-(Ethyl{[5-(3-methylphenyl)-1,2,4-oxadiazol-3-yl]methyl}amino)-4-methyl-4H-1,2,4-triazol-3-yl]-1-methylpyridin-2(1H)-one | 72% 0.1 g |

| | |
|---|---|
| ¹H NMR | (400 MHz, CDCl₃): δ 7.87 (s, 1H), 7.85 (m, 1H), 7.33-7-40 (m, 3H), 6.78 (dd, 1H), 6.73 (d, 1H), 4.56 (s, 2H), |
| | 3.70 (s, 3H), 3.54 (s, 3H), 3.37 (q, 2H), 2.39 (s, 3H), 1.18 (t, 3H) |

### Example 9

### 6-Oxo-1,6-dihydropyrimidine-4-carbohydrazide

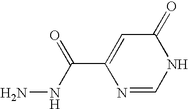

The subtitle compound of step 9A was stirred as a slurry in anhydrous methanol and hydrazine monohydrate (3 eq.) was added. The solid first dissolved but within 5 minutes the product started to precipitate. More methanol was added and the slurry was stirred at it overnight, filtered, washed with methanol, and dried in vacuo to give the title product (91%).

¹H NMR (400 MHz, DMSO-d6): δ 12.36 (broad s, 1H), 9.88 (s, 1H), 8.23 (s, 1H), 6.67 (s, 1H), 4.67 (s, 2H).

### Step 9A: Methyl 6-oxo-1,6-dihydropyrimidine-4-carboxylate

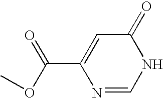

To 6-oxo-1,6-dihydropyrimidine-4-carboxylic acid (36.0 g, 257 mmol) in MeOH (360 mL) was dropwise added chlorotrimethylsilane (56 g, 554 mmol) and then stirred for 8 h at room temperature. The solvent was evaporated off and the solid was refluxed with 200 mL MeOH for 30 minutes. The reaction mixture was cooled, the precipitated solid was filtered off and washed with a small amount of MeOH and dried under vacuum at 35° C. to afford 27.9 g (70%) of the subtitle compound.

¹H NMR (300 MHz, DMSO-d6): δ (ppm) 12.50 (broad s, 1H), 8.23 (s, 1H), 6.83 (s, 1H), 3.80 (s, 3H).

### Example 10

### 6-Oxo-1,6-dihydropyridazine-4-carbohydrazide

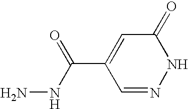

The compound of step 10C was heated with hydrazin hydrate (1.2 eq.) at 78° C. overnight. The reaction mixture was cooled and concentrated in vacuo. The residue was triturated with EtOAc, filtered and dried to give the title product (99%).

¹H NMR (400 MHz, DMSO-d6): δ 8.05 (d, 1H), 7.09 (d, 1H), 6.40 (broad s, 4H).

### Step 10A: 5-Methylpyridazin-3(2H)-one

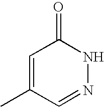

The 4,4-dimethoxy-3-methyl-but-2-enoic acid ethyl ester (Qi-Ying Au, Pankaj D. Rege, and E. J. Corey, J. Am. Chem. Soc., 2004, 126, 5984) (82 g, 440 mmol) was mixed with hydrazine hydrate (50 g, 999 mmol) at room temperature. The mixture was heated at 60° C. for 4 h. After evaporation of solvents the oil residue was further dried in vacuo. To the resulting residue was added 6 M aq. HCl. The mixture was heated at 60° C. for 5 h. The solvents were removed in vacuo. To the residue was added MeOH three times, followed by concentration in vacuo. The resulting residue was treated with dry EtOH followed by filtration to remove the solids. The filtrate was concentrated in vacuo. To the resulting residue was added dry IPA and 20 g anhydrous K₂CO₃. The mixture was heated for 20 min at 60° C. After filtration, and removal of solvents in vacuo, the residue was purified with flash chromatography using DCM: MeOH: Et₃N (10:1:0.3) to give the subtitle compound (13.4 g, 28%).

¹H NMR (400 MHz, MeOH-d4): d 2.24 (s, 3H), 6.73 (s, 1H), 7.82 (s, 1H).

### Step 10B: 6-Oxo-1,6-dihydropyridazine-4-carboxylic acid

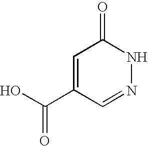

To a stirred solution of the subtitle compound of Step 10A (4.4 g, 40 mmol) in concentrated sulphuric acid (80 mL), potassium dichromate (18 g, 61 mmol) was added in small quantities at 50-60° C. as a finely ground powder. The starting material was added to the mixture within 20 min. Stirring was continued for 10 min at 60° C., the viscous green mixture was poured on crushed ice. The solids were filtered off and washed with cold water. After drying in vacuo the subtitle compound was isolated (4.5 g, 77%).

¹H NMR (400 MHz, DMSO-d6): δ 7.22 (s, 3H), 8.13 (s, 1H), 13.38 (s, broad, 1H).

### Step 10C: Ethyl 6-oxo-1,6-dihydropyridazine-4-carboxylate

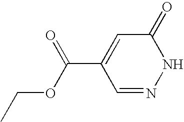

The subtitle compound of step 10B was dissolved in EtOH (10 mL) and concentrated H₂SO₄ (4.2 mL) was added and then heated at reflux for 5 hours. The reaction mixture was cooled, concentrated in vacuo and basified with saturated Na₂CO₃. After filtration, the aqueous phase was extracted with ethyl acetate, dried over anhydrous Na₂SO₄, filtered and concentrated to give the subtitle compound (83%).

¹H NMR (400 MHz, MeOH-d4): δ 8.27 (d, 1H), 7.42 (d, 1H), 4.40 (q, 2H), 1.39 (t, 3H).

### Example 11.1

### Methyl N,N′-dimethylimidothiocarbamate

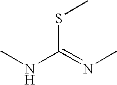

N,N′-Dimethylthiourea (29 g. 0.27 mol) was dissolved in acetone (300 mL) and cooled on ice bath. Methyliodide (27 mL, 0.44 mol) was added slowly. The ice bath was removed after 5 min. After 1 h at rt the precipitated solid was filtered off. The solids were dissolved in 1M NaOH (300 mL). Extracted with DCM (500 mL). The organic phase was passed through a phase separator and concentrated in vacuo to give the title compound that was used without further purifications (26 g, 80%).

¹H NMR (600 MHz, MeOH-d4): δ 2.85 (s, 6H), 2.37 (s, 3H)

The following compound was synthesised in a similar manner:

| Example | Structure | Name | Yield |
|---|---|---|---|
| 11.2 | 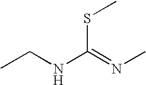 | Methyl N-ethyl-N′-methylimidothio-carbamate | 88%58 g |

| | |
|---|---|
| ¹H NMR | (400 MHz, DMSO-d6): δ 8.98 (s, 1H), 8.66 (s, 1H), 3.36 (m, |
| | 2H), 2.92 (m, 3H), 2.61 (s, 3H), 1.16 (m, 3H) |

### Example 12.1

### 1-methyl-4-[4-methyl-5-(methylamino)-4H-1,2,4-triazol-3-yl]pyridin-2(1H)-one

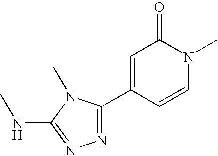

The title compound of Example 11.1 (1.5 g, 13 mmol) was slurried in DMSO (5 mL) and 1-Methyl-2-oxo-1,2-dihydro-pyridine-4-carboxylic acid hydrazide (WO 2008/041075, Example 31.1) (2.3 g, 14 mmol) was added. After heating to 80° C. and leaving the mixture over night, a clear solution was obtained. The heating was stopped after another hour and the reaction mixture was cooled on ice. The white solid was filtered off and washed with Et₂O. Freeze drying yielded the title compound as a white solid (1.6 g, 59%).

¹H NMR (400 MHz, D₂O): δ 7.68 (d, 1H), 6.68 (s, 1H), 6.61 (d, 1H), 3.51 (s, 3H), 3.34 (s, 3H), 2.81 (s, 3H).

The following compounds were synthesised in a similar manner:

| Example | Structure | Name | Yield |
|---|---|---|---|
| 12.2 | 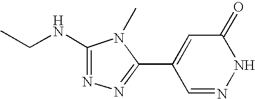 | 5-[5-(Ethylamino)-4-methyl-4H-1,2,4-triazol-3-yl]pyridazin-3(2H)-one | 62%7.4 gFrom Example 10and Example 11.2 |
| ¹H NMR | (400 MHz, DMSO-d6): δ 13.09 (s, 1H), 8.21 (d, 1H), 7.03 (d, 1H), 6.42 (t, 1H), 3.47 (s, 3H), | | |
| | 3.28 (q, 2H)*, 1.17 (t, 3H). | | |
| 12.3 | 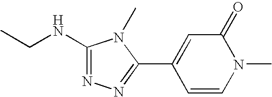 | 4-[5-(Ethylamino)-4-methyl-4H-1,2,4-triazol-3-yl]-1-methylpyridin-2(1H)-one | 60%3.2 gFrom Example 31.1in WO 2008/041075and Example 11.2 |
| ¹H NMR | (400 MHz, DMSO-d6): δ 7.73 (d, 1H), 6.59 (d, 1H), 6.52 (dd, 1H), 6.25 (t, 1H), 3.41 (s, 6H), | | |
| | 3.25 (dq, 2H), 1.17 (t, 3H) | | |
| 12.4 | 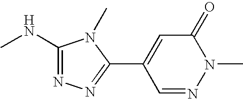 | 2-Methyl-5-[4-methyl-5-(methylamino)-4H-1,2,4-triazol-3-yl]pyridazin-3(2H)-one | 34%1.3 gFrom Example 21.8in US 2007/0259862and Example 11.1 |

| | |
|---|---|
| ¹H NMR | (400 MHz, MeOH-d4): δ 8.30 (d, 1H), 7.17 (d, 1H), 3.81 (s, 3H), 3.54 (s, 3H), 3.54 (s, 3H) |

*overlaps with residual solvent peak.

### Example 13

### [5-(3-Methylphenyl)-1,2,4-oxadiazol-3-yl]methanol

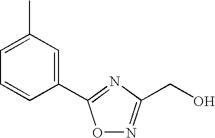

The material obtained in Step 13D was dissolved in DMSO (100 mL). Sulfuric acid (11.2 g, 114 mmol) was added over 10 seconds. The mixture was heated at 80° C. for 1 day until LCMS did not show M+18 intermediate peak. To the mixture was added n-heptane (200 mL). The DMSO layer was partitioned with DCM and aq. saturated NaHCO₃. The organic layer was washed with water and brine, dried (MgSO₄), followed by removal of solvents in vacuo to give a dry residue, which was purified by HPFC (Biotage 40+silica column) using a linear gradient EtOAC in heptane to elute the title product (7 g, 14% in 5 steps).

¹H NMR (400 MHz, CDCl₃): δ 7.96 (s, 1H), 7.94 (m, 1H), 7.42 (m, 2H), 4.87 (s, 2H), 2.45 (s, 3H).

### Step 13A: 2-{[tert-Butyl(dimethyl)silyl]oxy}acetamide

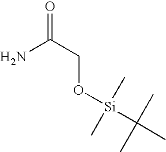

Under an atmosphere of nitrogen in a 1 L reactor which was equipped with overhead stirring was added a solution of 2-hydroxy-acetamide (20.5 g, 273 mmol) and pyridine (80.9 mL, 1002 mmol) in DMF (60 mL) at 25° C. The mixture was stirred at 25° C. for 30 min. To the mixture was added a solution of 50% TBDMSCl solution in toluene (100 g, 50%, 333 mmol) in MTBE (200 mL) within 70 min at 25° C. 3.5 h later, the reaction mixture was cooled to 10° C.

### Step 13B: {[tert-Butyl(dimethyl)silyl]oxy}acetonitrile

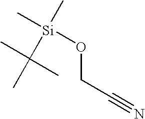

To the mixture obtained in Step 13A was added trifluoroacetic anhydride (45 mL, 323 mmol) during 35 min. The reaction mixture was stirred at 10° C. for 1 h. To the mixture was added water (200 mL) in 5 min. The temperature went up to 25° C. To the organic phase layer was added a solution of NaHCO₃ (10 g, 120 mmol) in water (110 mL). The mixture was stirred for 10 minutes and the layers were allowed to separate. The organic layer, in which contained the product, was used for the next step without any further actions.

### Step 13C: (1Z)-2-{[tert-Butyl(dimethyl)silyl]oxy}-N′-hydroxyethanimidamide

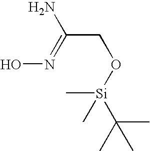

The solution obtained in Step 13B was heated to 55° C. and 50% aquous hydroxylamine (40 g, 606 mmol) was added during 80 min. To the mixture was added MTBE (200 mL). The organic layer was washed three time with water. A small amount solution was concentrated to dryness for NMR measurement. To the organic layer was added acetone (50 mL). The mixture was used for next step without any further actions.

¹H NMR (400 MHz, CDCl₃): δ 4.86 (bs, 2H), 4.14 (s, 2H), 0.88 (s, 9H), 0.07 (s, 6H); ¹³C NMR (400 MHz, CDCl₃): δ 153.5, 60.8, 25.9, 18.4, −5.3.

### Step 13D: (1Z)-2-{[tert-Butyl(dimethyl)silyl]oxy}-N′-{[(3-methylphenyl)carbonyl]-oxy}ethanimidamide

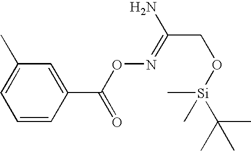

Triethylamine was added to the solution obtained in Step 13C at 0° C., followed by the addition of n7-toluoyl chloride (44.8 g, 290 mmol) in MTBE (20 mL) within 2.5 h. The reaction mixture was warmed to 20° C. To the mixture was added water (100 mL). The mixture was partitioned. The organic layer was washed with aq. NaHCO₃, followed with brine. The organic layer was concentrated at 40° C. under vacuum to an oily residue, which was used in the final step without further manipulations.

### Example 14

### 2-Methyl-5-[4-methyl-5-(methyl{(1S)-1-[5-(3-methylphenyl)isoxazol-3-yl]ethyl}amino)-4H-1,2,4-triazol-3-yl]pyridazin-3(2H)-one

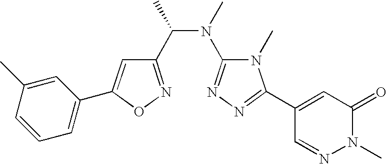

The title compound of Example 6.2 (0.63 g, 2.2 mmol) was dissolved in DMSO (11 mL) and the title compound of Example 12.4 (0.54 g, 2.5 mmol) was added, 2-methylpropan-2-olate (0.30 g, 3.1 mmol) was added and the mixture was stirred over night at room temp. The crude material was purified by reverse phase HPLC, Kromasil C8, 50.8×300 mm, 50 mL/min, linear gradient of 20% acetonitrile in water (0.2% formic acid) to 80% acetonitrile over 20 min, freeze dried to give the title compound (0.25 g, 27%).

¹H NMR (400 MHz, DMSO-d6): δ 8.31 (d, 1H), 7.68 (m, 2H), 7.43 (m, 1H), 7.32 (m, 1H), 7.21 (d, 1H), 7.13 (s, 1H), 4.84 (q, 1H), 3.71 (s, 3H), 3.70 (s, 3H), 2.76 (s, 3H), 2.39 (s, 3H), 1.60 (d, 3H).

### Biological Evaluation

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al. Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 cultured in a mixture of high glucose DMEM with Glutamax (31966-021)(500 mL), 10% dialyzed fetal bovine serum (Hyclone #SH30079.03) (56 mL), 200 μg/mL Hygromycin B (Invitrogen 45-0430, 50 mg/mL) (2.2 mL), 200 μg/mL Zeocin (Invitrogen #R250-01; 100 mg/mL) (1.1 mL) are seeded at a density of 100,000 cells per well on collagen coated clear bottom 96-well plates with black sides and cells were allowed to adhere over night before experiments. All assays are done in a buffer containing 146 mM NaCl, 5 mM KCl, 1 mM MgCl₂, 1 mM CaCl₂, 20 mM HEPES, 1 mg/mL glucose and 1 mg/mL BSA Fraction IV (pH 7.4). Cell cultures in the 96-well plates are loaded for 60 minutes in the above mentioned buffer containing 6 μM of the acetoxymethyl ester form of the fluorescent calcium indicator fluo-3 (Molecular Probes, Eugene, Oreg.) in 0.025% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.700 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 30 minutes, in dark at 25° C., interval separates the antagonist and agonist additions. The fluorescence signal is sampled 50 times at 1-second intervals followed by 3 samples at 5-second intervals immediately after each of the two additions. Responses are measured as the difference between the peak heights of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃). GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/mL glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 mL perchloric acid (5%) on ice, with incubation at 4° C. for at least 30 min. Samples are collected in 15 mL polypropylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 mL30 mM ammonium formate. Next, total inositol phosphates is eluted with 8 mL700 mM ammonium formate/100 mM formic acid and collected in scintillation vials. This eluate is then mixed with 8 mL of scintillant and [3H] inositol incorporation is determined by scintillation counting. The dpm counts from the duplicate samples are plotted and IC₅₀ determinations are generated using a linear least squares fitting program.

### ABBREVIATIONS

BSA Bovine Serum Albumin
CCD Charge Coupled Device
CRC Concentration Response Curve
DHPG 3,5-Dihydroxyphenylglycine
DPM Disintegrations per Minute
EDTA Ethylene Diamine Tetraacetic Acid
FLIPR Fluorometric Imaging Plate reader
SEEK GLAST-containing Human Embrionic Kidney
GLAST Glutamate/aspartate transporter
HEPES 4-(2-Hydroxyethyl)-1-piperazineethanesulfonic acid (buffer)
IP₃ Inositol triphosphate

Generally, the compounds were active in the assay above with IC₅₀ values less than 10 000 nM. In one aspect of the invention, the IC₅₀ value is less than 1 000 nM. In a further aspect of the invention, the IC₅₀ value is less than 100 nM.

### Determination of Brain to Plasma Ratio in Rat

Brain to plasma ratios are estimated in female Sprague Dawley rats. The compound is dissolved in water or another appropriate vehicle. For determination of brain to plasma ratio the compound is administrated as a subcutaneous, or an intravenous bolus injection, or an intravenous infusion, or an oral administration. At a predetermined time point after the administration a blood sample is taken with cardiac puncture. The rat is terminated by cutting the heart open, and the brain is immediately retained. The blood samples are collected in heparinized tubes and centrifuged within 30 minutes, in order to separate the plasma from the blood cells. The plasma is transferred to 96-well plates and stored at −20° C. until analysis. The brains are divided in half, and each half is placed in a pre-tarred tube and stored at −20° C. until analysis. Prior to the analysis, the brain samples are thawed and 3 mL/g brain tissue of distilled water is added to the tubes. The brain samples are sonicated in an ice bath until the samples are homogenized. Both brain and plasma samples are precipitated with acetonitrile. After centrifugation, the supernatant is diluted with 0.2% formic acid. Analysis is performed on a short reversed-phase HPLC column with rapid gradient elution and MSMS detection using a triple quadrupole instrument with electrospray ionisation and Selected Reaction Monitoring (SRM) acquisition. Liquid-liquid extraction may be used as an alternative sample clean-up. The samples are extracted, by shaking, to an organic solvent after addition of a suitable buffer. An aliquot of the organic layer is transferred to a new vial and evaporated to dryness under a stream of nitrogen. After reconstitution of the residuals the samples are ready for injection onto the HPLC column.

Generally, the compounds according to the present invention are peripherally restricted with a drug in brain over drug in plasma ratio in the rat of <0.5. In one embodiment, the ratio is less than 0.15.

### Determination of In Vitro Stability

Rat liver microsomes are prepared from Sprague-Dawley rats liver samples. Human liver microsomes are either prepared from human liver samples or acquired from BD Gentest. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding a 3.5 times volume of acetonitrile. The concentration of compound remaining in each of the collected samples is determined by means of LC-MS. The elimination rate constant (k) of the mGluR5 inhibitor is calculated as the slope of the plot of In[mGluR5 inhibitor] against incubation time (minutes). The elimination rate constant is then used to calculate the half-life (T ½) of the mGluR5 inhibitor, which is subsequently used to calculate the intrinsic clearance (CLint) of the mGluR5 inhibitor in liver microsomes as: CLint.=(In2×incubation volume)/(T ½×protein concentration)=μl/min/mg

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LBS) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 mL/kg) in a foreleg vein. Ten min after i.v. administration, a nutrient meal (10% peptone, 5% D-glucose, 5% Intralipid, pH 3.0) is infused into the stomach through the central lumen of the assembly at 100 mL/min to a final volume of 30 mL/kg. The infusion of the nutrient meal is followed by air infusion at a rate of 500 mL/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal≦2 s before its onset in which case the relaxation is classified as swallow-induced. The pressure difference between the LES and the stomach should be less than 2 ml-Hg, and the duration of the complete relaxation longer than 1 s.

### Specimen Results are Shown in the Following Table:

| | | Brain/Plasma Ratio |
|---|---|---|
| Example | FLIPR hmGluR5d (nM) | of compound in Rat |
| 4.1 | 41 | <0.01 |
| 4.2 | 31 | 0.02 |
| 4.3 | 21 | 0.015 |
| 4.4 | 13 | <0.01 |
| 4.5 | 77 | <0.01 |
| 4.6 | 45 | 0.01 |
| 4.7 | 53 | <0.015 |
| 4.8 | 112 | 0.22 |
| 4.9 | 53 | 0.035 |
| 4.10 | 65 | <0.01 |
| 4.11 | 43 | <0.01 |
| 4.12 | 71 | <0.01 |
| (−) 5.1 | 30 | <0.01 |
| (−) 5.2 | 111 | <0.01 |
| 8.1 | 10 | 0.03 |
| 8.2 | 20 | 0.16 |
| 8.3 | 117 | <0.01 |
| 8.4 | 43 | <0.01 |
| 8.5 | 52 | <0.01 |
| 8.6 | 75 | <0.01 |
| 14 | 12 | 0.07 |

1. A compound of formula (I) 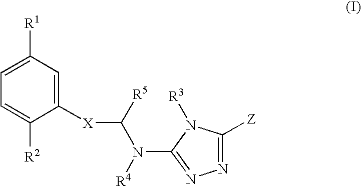
wherein
X is 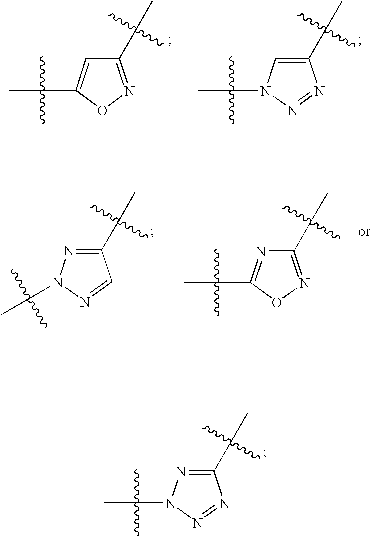
R¹ is
methyl, halogen or cyano;
R² is hydrogen or fluoro;
R³ is C₁-C₃ alkyl or cyclopropyl;
R⁴ is C₁-C₃ alkyl or cyclopropyl;
R⁵ is hydrogen, C₁-C₃ alkyl or cyclopropyl;
Z is 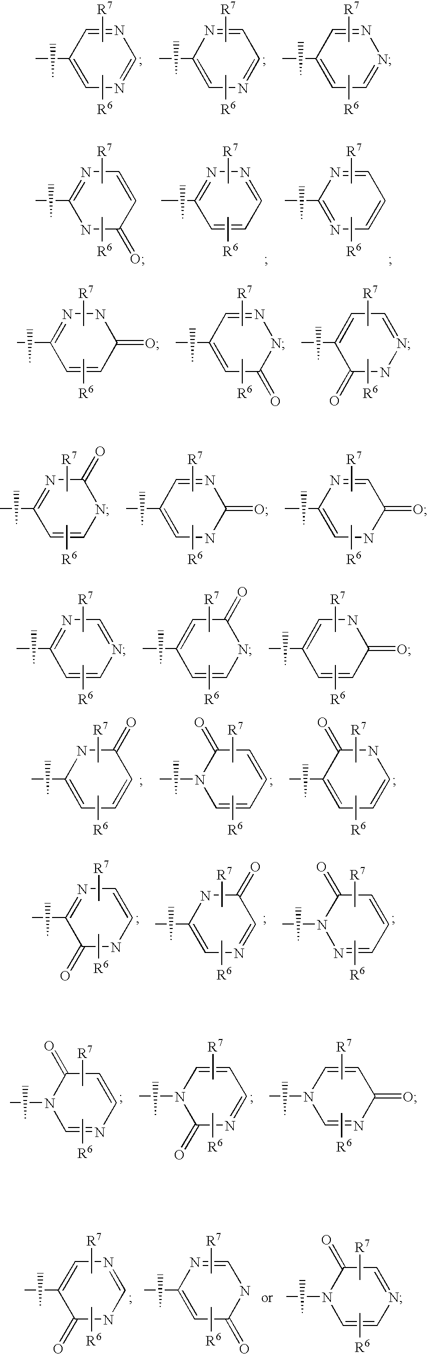
wherein
R⁶ is hydrogen, fluoro, C₁-C₃ alkyl or C₁-C₃ alkoxy;
R⁷ is hydrogen, fluoro, C₁-C₃ alkyl or C₁-C₃ alkoxy;
as well as pharmaceutically acceptable salts, hydrates, isoforms, tautomers and/or enantiomers thereof.

2. A compound according to claim 1, wherein R¹ is halogen.

3. A compound according to claim 2, wherein R¹ is chloro.

4. A compound according to claim 1, wherein R¹ is methyl.

5. A compound according to claim 1, wherein R² is hydrogen.

6. A compound according to claim 1, wherein R³ is methyl or cyclopropyl.

7. A compound according to claim 1, wherein R⁴ is methyl or ethyl.

8. A compound according to claim 1, wherein R⁵ is hydrogen or methyl.

9. A compound according to claim 1, wherein R⁶ is methyl and R⁷ is hydrogen.

10. A compound according to claim 1, wherein R⁶ is hydrogen and R⁷ is hydrogen.

11. A compound according to claim 1, wherein Z is

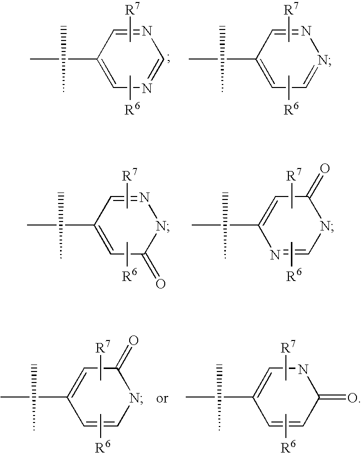

12. A compound according to claim 1, wherein
R¹ is halogen;
R² is hydrogen;
R³ is methyl or cyclopropyl;
R⁴ is methyl or ethyl;
R⁵ is hydrogen or methyl;
R⁶ is hydrogen or methyl;
R⁷ is hydrogen or methyl;
X is 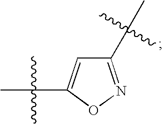
Z is 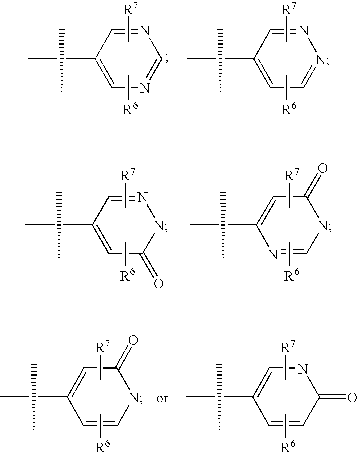
as well as pharmaceutically acceptable salts, hydrates, isoforms, tautomers and/or enantiomers thereof.

13. A compound according to claim 1, wherein
R¹ is methyl or halogen;
R² is hydrogen;
R³ is methyl or cyclopropyl;
R⁴ is methyl or ethyl;
R⁵ is hydrogen or methyl;
R⁶ is hydrogen or methyl;
R⁷ is hydrogen or methyl;
X is 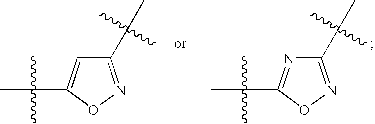
Z is 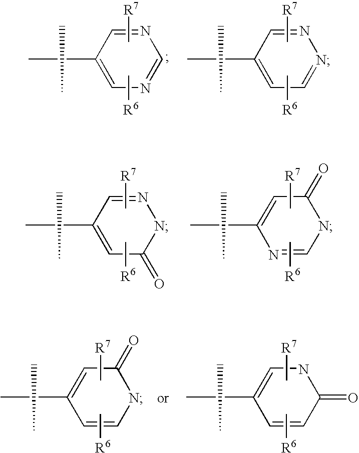
as well as pharmaceutically acceptable salts, hydrates, isoforms, tautomers and/or enantiomers thereof.

14. A compound according to claim 1 selected from
5-{5-[{[5-(3-Chlorophenyl)isoxazol-3-yl]methyl}(cyclopropyl)amino]-4-methyl-4H-1,2,4-triazol-3-yl}pyridazin-3(2H)-one;
4-{5-[{[5-(3-Chlorophenyl)isoxazol-3-yl]methyl}(ethyl)amino]-4-methyl-4H-1,2,4-triazol-3-yl}pyridin-2(1H)-one;
5-{5-[{[5-(3-Chlorophenyl)isoxazol-3-yl]methyl}(ethyl)amino]-4-methyl-4H-1,2,4-triazol-3-yl}pyridazin-3 (2H)-one;
6-{5-[{[5-(3-chlorophenyl)isoxazol-3-yl]methyl}(methyl)amino]-4-ethyl-4H-1,2,4-triazol-3-yl}pyrimidin-4(3H)-one;
6-{5-[{[5-(3-chlorophenyl)isoxazol-3-yl]methyl}(methyl)amino]-4-cyclopropyl-4H-1,2,4-triazol-3-yl}pyrimidin-4(3H)-one;
5-[5-(Ethyl{[5-(3-methylphenyl)isoxazol-3-yl]methyl}amino)-4-methyl-4H-1,2,4-triazol-3-yl]pyridazin-3 (2H)-one;
6-[4-Ethyl-5-(methyl {[5-(3-methylphenyl)isoxazol-3-yl]methyl}amino)-4H-1,2,4-triazol-3-yl]pyrimidin-4(3H)-one;
4-{5-[{1-[5-(3-Chlorophenyl)isoxazol-3-yl]ethyl}(methyl)amino]-4-methyl-4H-1,2,4-triazol-3-yl}-1-methylpyridin-2(1H)-one;
4-[5-(Ethyl{[5-(3-methylphenyl)isoxazol-3-yl]methyl}amino)-4-methyl-4H-1,2,4-triazol-3-yl]-1-methylpyridin-2(1H)-one;
4-[5-(Ethyl{[5-(3-methylphenyl)isoxazol-3-yl]methyl}amino)-4-methyl-4H-1,2,4-triazol-3-yl]pyridin-2(1H)-one;
4-{5-[{[5-(3-Chlorophenyl)isoxazol-3-yl]methyl}(ethyl)amino]-4-methyl-4H-1,2,4-triazol-3-yl}-1-methylpyridin-2(1H)-one,
(−)-5-[4-Methyl-5-(methyl{(1S)-1-[5-(3-methylphenyl)isoxazol-3-yl]ethyl}amino)-4H-1,2,4-triazol-3-yl]pyridazin-3(2H)-one;
(−)-4-[4-Methyl-5-(methyl{(1S)-1-[5-(3-methylphenyl)isoxazol-3-yl]ethyl}amino)-4H-1,2,4-triazol-3-yl]pyridin-2(1H)-one;
5-{5-[{1-[5-(3-Chlorophenyl)isoxazol-3-yl]ethyl}(cyclopropyl)amino]-4-methyl-4H-1,2,4-triazol-3-yl}pyridazin-3(2H)-one;
1-Methyl-4-[4-methyl-5-(methyl{(1S)-1-[5-(3-methylphenyl)isoxazol-3-yl]ethyl}amino)-4H-1,2,4-triazol-3-yl]pyridin-2(1H)-one;
5-{5-[{[5-(3-Chlorophenyl)-1,2,4-oxadiazol-3-yl]methyl}(ethyl)amino]-4-methyl-4H-1,2,4-triazol-3-yl}pyridazin-3(2H)-one;
5-[5-(Ethyl{[5-(3-methylphenyl)-1,2,4-oxadiazol-3-yl]methyl}amino)-4-methyl-4H-1,2,4-triazol-3-yl]pyridazin-3(2H)-one;
4-[5-(Ethyl {[5-(3-methylphenyl)-1,2,4-oxadiazol-3-yl]methyl}amino)-4-methyl-4H-1,2,4-triazol-3-yl]-1-methylpyridin-2(1H)-one; and
2-Methyl-5-[4-methyl-5-(methyl{(1S)-1-[5-(3-methylphenyl)isoxazol-3-yl]ethyl}amino)-4H-1,2,4-triazol-3-yl]pyridazin-3(2H)-one;
as well as pharmaceutically acceptable salts, hydrates, isoforms, tautomers and/or enantiomers thereof.

15. A compound according to claim 1 for use in therapy.

16. A pharmaceutical composition comprising a compound according to claim 1 as an active ingredient, together with a pharmacologically and pharmaceutically acceptable carrier.

17. Use of a compound according to claim 1, or a pharmaceutically acceptable salt or an optical isomer thereof, for the manufacture of a medicament for the inhibition of transient lower esophageal sphincter relaxations.

18. Use of a compound according to claim 1, or a pharmaceutically acceptable salt or an optical isomer thereof, for the manufacture of a medicament for treatment or prevention of gastroesophageal reflux disease.

19. Use of a compound according to claim 1, or a pharmaceutically acceptable salt or an optical isomer thereof, for the manufacture of a medicament for treatment or prevention of pain.

20. Use of a compound according to claim 1, or a pharmaceutically acceptable salt or an optical isomer thereof, for the manufacture of a medicament for treatment or prevention of anxiety.

21. Use of a compound according to claim 1, or a pharmaceutically acceptable salt or an optical isomer thereof for the manufacture of a medicament for treatment or prevention of irritable bowel syndrome (IBS).

22. A method for the inhibition of transient lower esophageal sphincter relaxations wherein an effective amount of a compound according to claim 1 is administered to a subject in need of such inhibition.

23. A method for the treatment or prevention of gastroesophageal reflux disease, wherein an effective amount of a compound according to claim 1 is administered to a subject in need of such treatment or prevention.

24. A method for the treatment or prevention of pain, wherein an effective amount of a compound according to claim 1 is administered to a subject in need of such treatment or prevention.

25. A method for the treatment or prevention of anxiety, wherein an effective amount of a compound according to claim 1 is administered to a subject in need of such treatment or prevention.

26. A method for the treatment or prevention of irritable bowel syndrome (IBS), wherein an effective amount of a compound according to claim 1 is administered to a subject in need of such treatment or prevention.

27. A combination comprising (i) at least one compound according to claim 1 and (ii) at least one acid secretion inhibiting agent.

28. A combination according to claim 27 wherein the acid secretion inhibiting agent is selected from cimetidine, ranitidine, omeprazole, esomeprazole, lansoprazole, pantoprazole, rabeprazole or leminoprazole.

29. A compound selected from
N-{[5-(3-Chlorophenyl)isoxazol-3-yl]methyl}cyclopropanamine;
N-{[5-(3-Chlorophenyl)isoxazol-3-yl]methyl}ethanamine;
1-[5-(3-Chlorophenyl)isoxazol-3-yl]-N-methylethanamine;
N-{L[5-(3-Methylphenyl)isoxazol-3-yl]methyl}ethanamine;
N-Methyl-1-[5-(3-methylphenyl)isoxazol-3-yl]methanamine;
N-{1-[5-(3-Chlorophenyl)isoxazol-3-yl]ethyl}cyclopropanamine;
(1S)-N-Methyl-1-[5-(3-methylphenyl)isoxazol-3-yl]ethanamine;
1-{[5-(3-Chlorophenyl)isoxazol-3-yl]methyl}-1-cyclopropyl-3-methylthiourea;
1-{[5-(3-Chlorophenyl)isoxazol-3-yl]methyl}-1-ethyl-3-methylthiourea;
1-{[5-(3-Chlorophenyl)isoxazol-3-yl]methyl}-3-ethyl-1-methylthiourea;
1-{[5-(3-Chlorophenyl)isoxazol-3-yl]methyl}-3-cyclopropyl-1-methylthiourea;
1-{1-[5-(3-Chlorophenyl)isoxazol-3-yl]ethyl}-1,3-dimethylthiourea;
1-Ethyl-3-methyl-1-{[5-(3-methylphenyl)isoxazol-3-yl]methyl}thiourea;
3-Ethyl-1-methyl-1-{[5-(3-methylphenyl)isoxazol-3-yl]methyl}thiourea;
1-{1-[5-(3-Chlorophenyl)isoxazol-3-yl]ethyl}-1-cyclopropyl-3-methylthiourea;
1,3-Dimethyl-1-{(1S)-1-[5-(3-methylphenyl)isoxazol-3-yl]ethyl}thiourea;
Methyl N-{[5-(3-chlorophenyl)isoxazol-3-yl]methyl}-N-cyclopropyl-N′-methylimidothiocarbamate;
Methyl N-{[5-(3-chlorophenyl)isoxazol-3-yl]methyl}-N-ethyl-N′-methylimidothiocarbamate;
Methyl N-{[5-(3-chlorophenyl)isoxazol-3-yl]methyl}-N′-ethyl-N-methylimidothiocarbamate;
Methyl N-{[5-(3-chlorophenyl)isoxazol-3-yl]methyl}-N′-cyclopropyl-N-methylimidothiocarbamate;
Methyl N-{1-[5-(3-chlorophenyl)isoxazol-3-yl]ethyl}-N,N′-dimethylimidothiocarbamate
Methyl N-ethyl-N′-methyl-N-{[5-(3-methylphenyl)isoxazol-3-yl]methyl}imidothiocarbamate;
Methyl N′-ethyl-N-methyl-N-{[5-(3-methylphenyl)isoxazol-3-yl]methyl}imidothiocarbamate;
Methyl N-{1-[5-(3-chlorophenyl)isoxazol-3-yl]ethyl}-N-cyclopropyl-N′-methylimidothiocarbamate;
Methyl N,N′-dimethyl-N-{(1S)-1-[5-(3-methylphenyl)isoxazol-3-yl]ethyl}imidothiocarbamate;
(1R)-1-[5-(3-Methylphenyl)isoxazol-3-yl]ethyl methanesulfonate;
[5-(3-methylphenyl)-1,2,4-oxadiazol-3-yl]methyl methanesulfonate
N,4-Dimethyl-5-pyrimidin-5-yl-4H-1,2,4-triazol-3-amine;
N,4-Dimethyl-5-pyridazin-4-yl-4H-1,2,4-triazol-3-amine;
1-Methyl-4-[4-methyl-5-(methylamino)-4H-1,2,4-triazol-3-yl]pyridin-2(1H)-one;
5-[5-(Ethylamino)-4-methyl-4H-1,2,4-triazol-3-yl]pyridazin-3(2H)-one;
4-[5-(Ethylamino)-4-methyl-4H-1,2,4-triazol-3-yl]-1-methylpyridin-2(1H)-one;
2-Methyl-5-[4-methyl-5-(methylamino)-4H-1,2,4-triazol-3-yl]pyridazin-3(2H)-one;
5-(3-Methylphenyl)-1,2,4-oxadiazol-3-yl]methanol;
2-{[tert-Butyl(dimethyl)silyl]oxy}acetamide;
[tert-Butyl(dimethyl)silyl]oxy}acetonitrile;
(1Z)-2-{[tert-Butyl(dimethyl)silyl]oxy}-N′-hydroxyethanimidamide; and
(1Z)-2-{[tert-Butyl(dimethyl)silyl]oxy}-N′-{[(3-methylphenyl)carbonyl]oxy}ethanimidamide.
